(19) United States
(12) Patent Application Publication
(10) Pub. No.: US 2004/0018228 A1
(43) Pub. Date: Jan. 29, 2004

(54) Compositions and methods for reducing scar tissue formation

(75) Inventors: Robert E. Fischell, Dayton, MD (US); Tim A. Fischell, Kalamazoo, MI (US); Sarah T. Fischell, Fair Haven, NJ (US); Clayton MacKenzie Waldorf, Richland, MI (US)

Correspondence Address: Peter G. Carroll, MEDLEN & CARROLL, LLP, Suite 350, 101 Howard Street, San Francisco, CA, 94105, US

(73) Assignee: Afmedica, Inc., Kalamazoo, MI

(21) Appl. No.: 10/431,701

(22) Filed: May 7, 2003

Related U.S. Application Data
(63) Continuation-in-part of application No. 10/351,207, filed on Jan. 24, 2003.
Application No. 10/351,207 is a continuation of application No. 09/772,693, filed on Jan. 31, 2001, now Pat. No. 6,534,693.
Application No. 09/772,693 is a continuation-in-part of application No. 09/705,999, filed on Nov. 6, 2000, now abandoned.

(51) Int. Cl. A61K 31/785; A61K 31/715; A61K 9/127

(52) U.S. Cl. 424/450; 424/78.22; 514/54

(57) ABSTRACT

The present invention describes the application of sirolimus and analogs of sirolimus to treat wound healing and reduce scar tissue formation. Also contemplated are non-sirolimus compounds believed to interact with the mTOR protein that have similar effects. Specifically, various medium are contemplated to create, for example, microparticles, foams, gels, sprays and bioadhesives that may be administered during surgical procedures involving either open or closed surgical site. Coating medical devices for long-term implantation is contemplated as one method of use of the above compositions.

### FIELD OF INVENTION

This invention is related to the field of tissue healing and excess scar prevention by pharmacological activity. Specifically, this invention is related to the use of sirolimus, tacrolimus and analogs of sirolimus (i.e., rapamycin and derivatives thereof) to reduce and/or prevent post-surgical scar tissue formation and/or adhesions.

### BACKGROUND

Excess post-operative scar tissue formation, adhesions and blood vessel narrowing are major problems following abdominal, neurological, spinal, vascular, thoracic or other types of surgery using both classical open and arthroscopic/laparoscopic procedures.

Scar tissue forms as part of the natural healing process of an injury whereupon the body usually initiates a full and swift wound healing response resulting in reconstructed, repaired tissue. In certain instances, however, this normal healing process may result in excessive scar tissue.

Following some kinds of surgery or injury, excess scar tissue production is a major problem which influences the result of surgery and healing. In the eye, for example, post-operative scarring can determine the outcome of surgery. This is particularly the case in the blinding disease glaucoma, where several anti-scarring regimens are currently used to improve glaucoma surgery results, but are of limited use clinically because of severe complications. Other examples of excess scar tissue production negatively impacting the outcome of surgery include adhesion lysis surgery, angioplasty, spinal surgery, vascular surgery and heart surgery.

Previous attempts to solve problematic post-surgical scarring have used highly cytotoxic mitosis inhibitors such as anthracycline, daunomycin, mitomycin C and doxorubin. Kelleher, U.S. Pat. No. 6,063,396. Similarly, intraluminal administration of cytostatic agents are reported to inhibit or reduce arterial restenosis. Kunz et al., U.S. Pat. No. 5,981,568.

The current state of the art is lacking in post-surgical and post-trauma treatments to significantly reduce the formation of scar tissue using compounds having a low medical risk and a high therapeutic benefit.

Definitions

The term “attached” as used herein, refers to any interaction between a medium or carrier and a compound. Attachment may be reversible or irreversible. Such attachment may be, but is not limited to, covalent bonding, ionic bonding, Van de Waal forces or friction, and the like. A compound is attached to a medium or carrier if it is impregnated, incorporated, coated, in suspension with, in solution with, mixed with, etc.

The term “contacting” as used herein, refers to any physical relationship between a biological tissue and a pharmaceutical compound attached to a medium. Such physical relationship may be, but is not limited to, spraying, layering, impregnation, interior placement into or exterior placement onto, and the like.

The term “wound” as used herein, denotes a bodily injury with disruption of the normal integrity of tissue structures. In one sense, the term is intended to encompass a “surgical site”. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc. Conditions related to wounds or sores which may be successfully treated according to the invention are skin diseases.

The term “surgical site” as used herein, refers to any opening in the skin or internal organs performed for a specific medical purpose. The surgical site may be “open” where medical personnel have direct physical access to the area of interest as in traditional surgery. Alternatively, the surgical site may be “closed” where medical personnel perform procedures using remote devices such as, but not limited to, catheters wherein fluoroscopes may be used to visualize the activities and; endoscopes (i.e., laparoscopes) wherein fiber optic systems may be used to visualize the activities. A surgical site may include, but is not limited to, organs, muscles, tendons, ligaments, connective tissue and the like.

The term “organ” as used herein, include, without limitation, veins, arteries, lymphatic vessels, esophagus, stomach, duodenum, jejunum, ileum, colon, rectum, urinary bladder, ureters, gall bladder, bile ducts, pancreatic duct, pericardial sac, peritoneum, and pleura.

The term “skin” is used herein, very broadly embraces the epidermal layer of the skin and, if exposed, also the underlying dermal layer. Since the skin is the most exposed part of the body, it is particularly susceptible to various kinds of injuries such as, but not limited to, ruptures, cuts, abrasions, burns and frostbites or injuries arising from the various diseases.

The term “anastomosis” as used herein, refers to a surgical procedure where two vessels or organs, each having a lumen, are placed in such proximity that growth is stimulated and the two vessels or organs are joined by forming continuous tissue. Preferably, the bodily organs to be joined are veins, arteries and portions of the intestinal tract. Most preferably, the organs to be joined are arteries. One of skill in the art will recognize that an anastomosis procedure contemplated by the present invention is amenable to use not only in all areas of vascular surgery but also in other surgical procedures for joining organs. Examples of anastomoses that can be performed include, but are not limited to, arterial anastomosis, venous anastomosis, arterio-venous anastomosis, anastomosis of lymphatic vessels, gastroesophageal anastomosis, gastroduodenal anastomosis, gastrojejunal anastomosis, anastomosis between and among the jejunum, ileum, colon and rectum, ureterovesicular anastomosis, anastomosis of the gall bladder or bile duct to the duodenum, and anastomosis of the pancreatic duct to the duodenum. In addition, an anastomosis may join an artifical graft to a bodily organ that has a lumen. In one embodiment, the present invention contemplates contacting a medium with an arterio-venous anastomosis of a patient, wherein said patient exhibits symptoms of end stage renal disease and is undergoing dialysis.

The term “communication” as used herein, refers to the ability of two organs to exchange body fluids by flowing or diffusing from one organ to another in the manner typically associated with the organ pair that has is been joined. Examples of fluids that might flow through an anastomosis include, but are not limited to, liquid and semi-solids such as blood, urine, lymphatic fluid, bile, pancreatic fluid, ingesta and purulent discharge.

The term “medium” as used herein, refers to any material, or combination of materials, which serve as a carrier or vehicle for delivering of a compound to a treatment point (e.g., wound, surgical site etc.). For all practical purposes, therefore, the term “medium” is considered synonymous with the term “carrier”. In one embodiment, a medium comprises a carrier, wherein said carrier is attached to a drug or compound and said medium facilitates delivery of said carrier to a treatment point. In another embodiment, a carrier comprises an attached drug wherein said carrier facilitates delivery of said drug to a treatment point. Preferably, a medium is selected from the group consisting of foams, gels (including, but not limited to, hydrogels), xerogels, microparticles (i.e., microspheres, liposomes, microcapsules etc.), bioadhesives and liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of drugs over a period of time lasting approximately from 1 day to 6 months.

The term “xerogel” as used herein, refers to any device comprising a combination of silicone and oxygen having a plurality of air bubbles and an entrapped compound. The resultant glassy matrix is capable of a controlled release of an entrapped compound during the dissolution of the matrix.

The term “material” as used herein refers to any chemical that is useful in the creation of a medium. For example, a liposome medium is comprised of a phospholipid material; a microparticle or hydrogel medium is comprised of a polymer material, wherein said polymer material is exemplified by poly(lactide-co-glycolide) copolymers and hyaluronic acid.

The term “reduction in scar tissue formation” as used herein refers to any tissue response that reflects an improvement in wound healing. Specifically, improvement in conditions such as, but not limited to, hyperplasia or adverse reactions to post-cellular trauma are contemplated. It is not contemplated that all scar tissue must be avoided. It is enough if the amount of scarring or hyperplasia is reduced as compared to untreated patients.

The term “foam” as used herein, refers to a dispersion in which a large proportion of gas, by volume, is in the form of gas bubbles and dispersed within a liquid, solid or gel. The diameter of the bubbles are usually relatively larger than the thickness of the lamellae between the bubbles.

The term “gel” as used herein, refers to any material forming, to various degrees, a medium viscosity liquid or a jelly-like product when suspended in a solvent. A gel may also encompass a solid or semisolid colloid containing a certain amount of water. These colloid solutions are often referred to in the art as hydrosols. One specific type of gel is a hydrogel. The term “hydrogel” as used herein, refers to any material forming, to various degrees, a jelly-like product when suspended in a solvent, typically water or polar solvents comprising such as, but not limited to, gelatin and pectin and fractions and derivatives thereof. Typically, a hydrogel is capable of swelling in water and retains a significant portion of water within its structure without dissolution. In one embodiment, the present invention contemplates a gel that is liquid at lower than body temperature and forms a firm gel when at body temperature.

The term “spray” as used herein, refers to any suspension of liquid or particles blown, ejected into, or falling through the air. Sprays can be jets of fine particles or droplets. A spray can be an aerosol.

An “aerosol” is herein defined as a suspension of liquid or solid particles of a substance (or substances) in a gas, such as, but not limited to dispersions. Aerosols may comprise solid or liquid dispersions. The present invention contemplates the generation of aerosols by both atomizers and nebulizers of various types. An “atomizer” is an aerosol generator without a baffle, whereas a “nebulizer” uses a baffle to produce smaller particles. In one embodiment, the present invention contemplates using the commercially available Aerogen™ aerosol generator which comprises a vibrational element and dome-shaped aperture plate with tapered holes. When the plate vibrates several thousand times per second, a micro-pumping action causes liquid to be drawn through the tapered holes, creating a low-velocity aerosol with a precisely defined range of droplet sizes. The Aerogen™ aerosol generator does not require propellant. “Baffling” is the interruption of forward motion by an object, i.e. by a “baffle.” Baffling can be achieved by having the aerosol hit the sides of the container or tubing. More typically, a structure (such as a ball or other barrier) is put in the path of the aerosol (See e.g. U.S. Pat. No. 5,642,730, hereby incorporated by reference). The present invention contemplates the use of a baffle in order to slow the speed of the aerosol as it exits the delivery device.

The term “compound” or “drug” as used herein, refers to any pharmacologically active substance capable of being administered which achieves a desired effect. Compounds or drugs can be synthetic or organic, proteins or peptides, oligonucleotides or nucleotides, polysaccharides or sugars. Compounds or drugs may have any of a variety of activities, which may be stimulatory or inhibitory, such as antibiotic activity, antiviral activity, antifungal activity, steroidal activity, cytotoxic, cytostatic, anti-proliferative, anti-inflammatory, analgesic or anesthetic activity, or can be useful as contrast or other diagnostic agents. In a preferred embodiment, the present invention contemplates compounds or drugs that are capable of binding to the mTOR protein and either reduce wound and post-surgical adhesions and/or reduce wound and post-surgical scarring. In another embodiment, the present invention contemplates compounds or drugs that are cytostatic and are believed to primarily act by interrupting the cell division cycle in the G0 or G1 stage, thus inhibiting proliferation without killing the cell. It is not intended that the term compound or drug refers to any non-pharmaceutically active material such as, but not limited to, polymers or resins intended for the creation of any one specific medium.

The term “rapamycin” as used herein refers to a compound represented by the drug sirolimus. Rapamycin is an antifungal antibiotic which may be naturally extracted from a streptomycetes, e.g., Streptomyces hygroscopicus, chemically synthesized or produced by genetic engineering cell culture techniques.

The term “analog” as used herein, refers to any compound having substantial structure-activity relationships to a parent compound such that the analog has similar biochemical activity as the parent compound. For example, sirolimus has many analogs that are substituted at either the 2-, 7- or 32- positions. One of skill in the art should understand that the term “derivative” is used herein interchangeably with term “analog”.

The term “administered” or “administering” a compound or drug, as used herein, refers to any method of providing a compound or drug to a patient such that the compound or drug has its intended effect on the patient. For example, one method of administering is by an indirect mechanism using a medical device such as, but not limited to a catheter, spray gun, syringe etc. A second exemplary method of administering is by a direct mechanism such as, oral ingestion, transdermal patch, topical, inhalation, suppository etc.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatiblity is evaluated according to the application for which it was designed: for example; a bandage is regarded a biocompable with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long term inflammatory effects such that the molecular structure has not been altered. In one sense, bioerosin represents the final stages of “biodegradation” wherein stable low molecular weight products undergo a final dissolution.

The term “bioresorbable” as used herein, refers to any material that is assimilated into or across bodily tissues. The bioresorption process may utilize both biodegradation and/or bioerosin.

The term “biostable” as used herein, refers to any material that remains within a physiological environment for an intended duration resulting in a medically beneficial effect.

The term “supplemental pharmaceutical compound” as used herein, refers to any medically safe compound administered as part of a medium as contemplated by this invention. Administration of a medium comprising a supplemental pharmaceutical compound includes, but is not limited to, systemic, local, implantation or any other means. A supplemental pharmaceutical compound may have activities similar to, or different from a compound capable being cytostatic or of binding to the mTOR protein. Preferably, supplemental pharmaceutical compounds include, but are not limited to, antiinflammatory drugs, corticosteriods, antithrombotics, antibiotics, antivirals, analgesics and anesthetics.

The term “complementary pharmaceutical compound” as used herein, refers to any medically safe compound administered separately from a medium as contemplated by this invention. Administration of a complementary pharmaceutical compound includes, but is not limited to, oral ingestion, transdermal patch, topical, inhalation, suppository etc. Preferably, complementary pharmaceutical compounds include, but are not limited to, sirolimus, tacrolimus, analogs of sirolimus, antiinflammatory drugs, corticosteroids, antithrombotics, antibiotics, antivirals, analgesics and anesthetics.

The term “colloidal system” or “colloid” as used herein, refers to a substance that consists of particles dispersed throughout another substance which are too small for resolution with an ordinary light microscope but are incapable of passing through a semipermeable membrane. It is not necessary for all three dimensions to be within the colloidal system: fibers may exhibit only two dimensions as a colloid, and thin films may have only a single dimension as a colloid. It is not necessary for the units of a colloidal system to be discrete: continuous network structures, the basic units of which are of colloidal dimensions also fall in this class (e.g. porous solids, gels and foams). A fluid colloidal system may be composed of two or more components and called a sol, e.g. a protein sol, a gold sol, an emulsion, a surfactant solution above the critical micelle concentration, or an aerosol. In a suspension solid particles are dispersed in a liquid; a colloidal suspension is one in which the size of the particles lies in the colloidal range.

The term “dose metering element” as used herein, is an element that controls the amount of compound administered. The element can, but need not, measure the amount of compound as it is administered. In a preferred embodiment, the element is characterized simply as a container of defined volume (e.g., a reservoir). In a preferred embodiment, the defined volume is filled by the manufacturer or hospital professional (e.g., nurse, pharmacist, doctor, etc.) and the entire volume is administered. In another embodiment, the reservoir is configured as a transparent or semi-transparent cylinder with visible measurement indicia (e.g. markings, numbers, etc.) and the filling is done to a desired point (e.g. less than the entire capacity) using the indicia as a guide.

The term “fluid driving element” as used herein, is an element that moves fluid in a direction along the device. In some embodiments, the fluid driving element comprises a plunger driven by compressed gas, said compressed gas stored in a canister. In other embodiments, it comprises a pump. In still other embodiments, it comprises a hand actuated plunger (in the manner of a syringe).

The term “patient” as used herein, is a human or animal and need not be hospitalized. For example, out-patients, persons in nursing homes are “patients.”

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a compound or drug to a patient during a medical procedure or therapy is contemplated herein as a medical device. “Direct medical implants” include, but are not limited to, urinary and intravascular catheters, dialysis shunts, wound drain tubes, skin sutures, vascular grafts and implantable meshes, intraocular devices, implantable drug delivery systems and heart valves, and the like. “Wound care devices” include, but are not limited to, general wound dressings, non-adherent dressings, burn dressings, biological graft materials, tape closures and dressings, and surgical drapes. “Surgical devices” include, but are not limited to, endoscope systems (i.e., catheters, vascular catheters, surgical tools such as scalpels, retractors, and the like) and temporary drug delivery devices such as drug ports, injection needles etc. to administer the medium. A medical device is “coated” when a medium comprising a cytostatic or antiproliferative drug (i.e., for example, sirolimus or an analog of sirolimus) becomes attached to the surface of the medical device. This attachment may be permanent or temporary. When temporary, the attachment may result in a controlled release of a cytostatic or antiproliferative drug.

The term “cytostatic” refers to any compound whose principal mechanism of antiproliferative action interferes with the progress of the cell cycle in the G0 or G1 phase. In one embodiment, sirolimus, tacrolimus or analogs of sirolimus are cytostatic and interfere with (i.e., stop) the cell cycle from progressing out of the G1 phase.

The term “endoscope” refers to any medical device that is capable of being inserted into a living body and used for tasks including, but not limited to, observing surgical procedures, performing surgical procedures, applying medium to a surgical site. An endoscope is illustrated by instruments including, but not limited to, an arthroscope, a laparoscope, hysteroscope, cytoscope, etc. It is not intended to limit the use of an endoscope to hollow organs. It is specifically contemplated that endoscopes, such as an arthroscope or a laparoscope is inserted through the skin and courses to a closed surgical site.

The term “liquid” as used herein, refers to a minimally viscous medium that is applied to a surgical site by methods including, but not limited to, spraying, pouring, squeezing, spattering, squirting, and the like.

The term “dispense as a liquid” as used herein, refers to spraying, pouring, squeezing, spattering, squirting, and the like.

The term “liquid administration” as used herein, refers to any method by which a medium comprises an ability to flow or stream, either in response to gravity or by pressure-induced force.

The term “liquid spray” as used herein, refers to a liquid administration comprising the generation of finely dispersed droplets in response to pressure-induced force, wherein the finely dispersed droplets settle onto a surgical site by gravity.

The term “pourable liquids” as used herein, refers to a liquid administration comprising the flowing or streaming of a low viscosity liquid in response to gravity. The present invention contemplates low viscosity liquids (at room temperature) ranging from between 1 and 15,000 centipoise, preferably between 1 and 500 centipoise (i.e., similar to saturated glucose solution) and more preferably between 1 and 250 centipoise (i.e., similar to motor oil).

The term “squeezable liquids” as used herein, refers to a liquid administration comprising the flowing or streaming of a high viscosity liquid in response to a pressure-induced force. The-present invention contemplates high viscosity liquids (at room temperature) ranging from between 5,000 and 100,000 centipoise, preferably between 25,000 and 50,000 centipoise (i.e., similar to mayonnaise), more preferably between 15,000 and 25,000 centipoise (i.e., similar to molten glass), and more preferably between 5,000 and 15,000 centipoise (i.e., similar to honey).

The term, “microparticle” as used herein, refers to any microscopic carrier to which a compound or drug may be attached. Preferably, microparticles contemplated by this invention are capable of formulations having controlled release properties.

The term “PLGA” as used herein, refers to mixtures of polymers or copolymers of lactic acid and glycolic acid. As used herein, lactide polymers are chemically equivalent to lactic acid polymer and glycolide polymers are chemically equivalent to glycolic acid polymers. In one embodiment, PLGA contemplates an alternating mixture of lactide and glycolide polymers, and is referred to as a poly(lactide-co-glycolide) polymer.

### SUMMARY

This invention is related to the field of tissue healing and scar prevention. In one embodiment, pharmaceutical compounds are used to reduce and/or prevent scar tissue formation. In another embodiment, sirolimus, tacrolimus and analogs of sirolimus (i.e., sirolimus and it's derivatives) are used to reduce and/or prevent post-surgical scar tissue formation. In another embodiment, compounds capable of interrupting the cell cycle at the G0 or G1 stage are used to reduce and/or prevent excess scar tissue. In another embodiment, compounds capable of binding to the mTOR protein are used to reduce and/or prevent scar tissue formation.

One aspect of the present invention contemplates a drug attached to a carrier, the drug being selected from the group consisting of sirolimus, tacrolimus, everolimus and the analogs and derivatives of the drug, the carrier onto which the drug is attached being selected from the group consisting of microparticles, gels, xerogels, bioadhesives, foams and liquids. In one embodiment the carrier comprises a biocompatible material. In another embodiment, the carrier comprises a biodegradable material. In one embodiment, the microparticles are selected from the group consisting of microspheres, microencapsulating particles, microcapsules and liposomes. In one embodiment, the microparticle comprises a polymer selected from the group consisting of poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedopoly(amino acids), polyhydroxybutrate-related copolymers, polyanhydrides, polymethylmethacrylate, poly(ethylene oxide), lecithin and phospholipids. In one embodiment the carrier comprises a material selected from the group consisting of gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof. In another embodiment, the carrier comprises a polymer selected from the group consisting of poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids. In one embodiment, the carrier releases said drug in a controlled release manner. In one embodiment, the carrier is colored. In one embodiment, the carrier further comprises a radio-opaque marker, wherein said marker is visualized by X-ray spectroscopy.

One aspect of the present invention contemplates a medium, comprising a compound selected from the group consisting of sirolimus, tacrolimus, analogs of sirolimus and pharmaceutically acceptable salts thereof, wherein said medium is selected from the group consisting of microparticles, gels, bioadhesives, hydrogels, xerogels, foams and combinations thereof. In one embodiment, said medium comprises a biocompatible material. In one embodiment, said medium comprises a biodegradable material. In one embodiment, said medium provides controlled release of said compound. In one embodiment, said microparticles are selected from the group consisting of microspheres, microencapsulating particles, microcapsules and liposomes. In one embodiment, the microparticle comprises a polymer selected from the group consisting of poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids. In one embodiment the medium comprises a material selected from the group consisting of gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof. In another embodiment, the medium comprises a polymer selected from the group consisting of poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids. In one embodiment, said medium is colored. In one embodiment, said medium further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said medium further comprises a supplemental pharmaceutical compound selected from the group consisting of antiinflammatory, corticosteriods, antithrombotics, antibiotics, antivirals, analgesics and anesthetics.

One aspect of the present invention contemplates a composition, comprising: a) a medium; and b) a compound attached to said medium, said compound selected from the group consisting of sirolimus, tacrolimus, analogs of sirolimus and pharmaceutically acceptable salts thereof. In one embodiment, said medium comprises a biocompatible material. In another embodiment, said medium comprises a biodegradable material. In one embodiment, said medium provides controlled release of said compound. In one embodiment, said medium is selected from the group consisting of a microparticles, liquids, foams, gels, hydrogels, xerogels and bioadhesives. In another embodiment, said medium is a spray. In one embodiment, said medium comprises a microparticle. In one embodiment, said microparticle is a microencapsulating particle. In one embodiment, said microencapsulating particle is selected from the group consisting of microcapsules, microspheres and liposomes. In one embodiment, said medium is colored. In one embodiment, said medium further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said composition further comprises antisense to c-myc. In another embodiment, said composition further comprises tumstatin. In one embodiment, said composition further comprises a supplemental pharmaceutical compound selected from the group consisting of antiinflammatory, corticosteriods, antithrombotics, antibiotics, antivirals, analgesics and anesthetics.

Another aspect of the present invention contemplates a composition, comprising: a) a microparticle; and b) a compound attached to said microparticle, said compound selected from the group consisting of sirolimus, tacrolimus, analogs of sirolimus and pharmaceutically acceptable salts thereof. In one embodiment, said microparticle comprises a biocompatible material. In one embodiment, said microparticle comprises a biodegradable material. In one embodiment, said microparticle is a microsphere. In one embodiment, said microparticle is a microencapsulating particle. In one embodiment, said medium provides controlled release of said compound. In one embodiment, said microencapsulating particle is selected from the group consisting of microcapsules and liposomes. In one embodiment, said microparticle is colored. In one embodiment, said microparticle further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said composition further comprises antisense to c-myc. In another embodiment, said composition further comprises tumstatin. In one embodiment, said composition further comprises, a supplemental pharmaceutical compound selected from the group consisting of antiinflammatory, corticosteriods, antithrombotics, antibiotics, antivirals, analgesics and anesthetics.

One aspect of the present invention contemplates a composition, comprising: a) a microparticle, wherein said microparticle encapsulates a compound selected from the group consisting of sirolimus, tacrolimus, analogs of sirolimus and pharmaceutically acceptable salts thereof; and b) a biocompatible and biodegradable material to which said microparticle is attached. In one embodiment, said microparticle is selected from the group consisting of microspheres, microcapsules and liposomes. In one embodiment, said microparticle provides controlled release of said compound. In one embodiment, said microparticle is clear. In another embodiment, said microparticle is colored. In one embodiment, said microparticle further comprises a radio-opaque marker, wherein said marker is visualized by X-ray spectroscopy. In one embodiment, said biocompatible and biodegradable material is selected from the group consisting of-polylactide-polyglycolide polymers, lactide/glycolide copolymers, poly(lactide-co-glycolide) polymers (i.e., PLGA), hyaluronic acid, modified polysaccharides and any other well known substance that is known to be both biocompatible and biodegradable. In one embodiment, said analog of sirolimus comprises a compound capable of binding to the mTOR protein. In one embodiment, said compound capable of binding to the mTOR protein is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said composition further comprises anti-sense to c-myc. In another embodiment, said composition further comprises tumstatin. In one embodiment, said microparticle further comprises a plurality of supplemental pharmaceutical compounds. In one embodiment, said supplemental pharmaceutical compound is selected from the group consisting of antiinflammatory, corticosteriods, antithrombotics, antibiotics, antivirals, analgesics and anesthetics.

Another aspect of the present invention is a composition, comprising: a) a biocompatible and biodegradable hydrogel; and b) a compound selected from the group consisting of sirolimus, tacrolimus, analogs of sirolimus and pharmaceutically acceptable salts thereof, wherein said compound is attached to said hydrogel. In one embodiment, said hydrogel comprises a material selected from the group consisting of gelatins, pectins, collagens, hemoglobins, carbohydrates, hyaluronic acid, cellulose esters, Carbopol®, synthetic polyvinylpyrrolidone, polyethyleneoxide, acrylate, and methacrylate and copolymers thereof. In one embodiment, said hydrogel provides controlled release of said compound. In one embodiment, said analog of sirolimus comprises a compound capable of binding to the mTOR protein. In one embodiment, said compound capable of binding to the mTOR protein is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said composition further comprises antisense c-myc. In another embodiment, said composition further comprises tumstatin. In one embodiment, said biodegradable and biocompatible hydrogel further comprises a plurality of supplemental pharmaceutical compounds. In one embodiment, said supplemental pharmaceutical compound is selected from the group consisting of antiinflammatory, corticosteriods, antithrombotics, antibiotics, antivirals, analgesics and anesthetics. In one embodiment, a cytostatic pharmaceutical compound is attached to a polymer medium that is incorporated into said hydrogel. In one embodiment, said polymer medium is biodegradable and has a different release rate and biodegradation characteristics than said hydrogel. In another embodiment, said polymer medium is selected from the group comprising polylactide-polyglycolide polymers, lactide/glycolide copolymers, poly(lactide-co-glycolide) polymers (i.e., PLGA), hyaluronic acid or other similar polymers. In one embodiment, said hydrogel comprises a microparticle incorporating a cytostatic drug,

Another aspect of the present invention contemplates a composition, comprising: a) a biocompatible bioadhesive; and b) a compound selected from the group consisting of sirolimus, everolimus, analogs of sirolimus and pharmaceutically acceptable salts thereof, wherein said compound is attached to said bioadhesive. In one embodiment, said bioadhesive is biodegradable. In one embodiment, said bioadhesive provides controlled release of said compound. In one embodiment, said bioadhesive comprises a material selected from the group consisting of fibrin, fibrinogen, calcium polycarbophil, polyacrylic acid, gelatin, carboxymethyl cellulose, natural gums such as karaya and tragacanth, algin, cyanoacrylates, chitosan, hydroxypropylmethyl cellulose, starches, pectins or mixtures thereof. In one embodiment, said bioadhesive further comprises a hydrocarbon gel base, wherein said base is composed of polyethylene and mineral oil. In one embodiment, said base has a preselected pH level, wherein said pH level maintains said base stability. In one embodiment, said analog of sirolimus comprises a compound capable of binding to the mTOR protein selected from the group consisting of tacrolimus, everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said composition further comprises antisense to c-myc. In another embodiment, said composition further comprises tumstatin. In one embodiment, said bioadhesive further comprises a plurality of supplemental pharmaceutical compounds. In one embodiment, said supplemental pharmaceutical compound is selected from the group consisting of antiinflammatory, corticosteriods, antithrombotics, antibiotics, antivirals, analgesics and anesthetics.

Another aspect of the present invention contemplates a gel, comprising a compound selected from the group consisting of sirolimus, everolimus, analogs of sirolimus and pharmaceutically acceptable salts thereof. In one embodiment, said gel comprises a hydrogel. In one embodiment, said gel provides controlled release of said compound. In one embodiment, said gel is colored. In one embodiment, said gel further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said gel further comprises antisense c-myc. In another embodiment, said gel further comprises tumstatin. In one embodiment, said gel further comprises a supplemental pharmaceutical compound selected from the group consisting of antiinflammatory, corticosteriods, antithrombotics, antibiotics, antivirals, analgesics and anesthetics. One embodiment contemplates a surgical device wherein at least a portion of said device comprises an attached gel comprising sirolimus and analogs of sirolimus.

Another aspect of the present invention contemplates a foam, comprising a compound selected from the group consisting of sirolimus, tacrolimus, analogs of sirolimus and pharmaceutically acceptable salts thereof. In one embodiment, said foam further comprises a xerogel. In one embodiment, said foam provides controlled release of said compound. In one embodiment, said foam is colored. In one embodiment, said foam further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said foam further comprises antisense c-myc. In another embodiment, said foam further comprises tumstatin. In one embodiment, said foam further comprises a supplemental pharmaceutical compound selected from the group consisting of antiinflammatory, corticosteriods, antithrombotics, antibiotics, antivirals, analgesics and anesthetics. One embodiment contemplates a surgical device wherein at least a portion of said device comprises an attached foam comprising sirolimus and analogs of sirolimus.

One aspect of the present invention contemplates a method, comprising: a) providing: i) a medium comprising a compound selected from the group consisting of sirolimus, tacrolimus, analogs of sirolimus and pharmaceutically acceptable salts thereof, wherein said medium is selected from the group consisting of microparticles, gels, xerogels, hydrogels, bioadhesives, foams and combinations thereof; and ii) a patient, wherein said patient has a surgical site; and b) contacting said surgical site with said medium. In one embodiment, said surgical site comprises a closed surgical site. In another embodiment, said surgical site comprises an open surgical site. In one embodiment, said medium of step a) is housed in a device capable of delivering said medium to said surgical site. In one embodiment, said device delivers said medium by brushing. In one embodiment, said device delivers said medium by liquid administration. In one embodiment, said liquid administration comprises a liquid spray. In one embodiment, said liquid spray in the form of an aerosol. In one embodiment, said liquid administration comprises a pourable liquid. In another embodiment, said liquid administration comprises a squeezable liquid. In one embodiment, said device comprises a catheter. In one embodiment, said device is configured for endoscopic surgery. In one embodiment, said medium comprises a biocompatible material. In one embodiment, said medium comprises a biodegradable material. In one embodiment, said microparticles are selected from the group consisting of microspheres, microencapsulating particles, microcapsules and liposomes. In one embodiment, said medium is colored. In one embodiment, said medium further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said method further comprises administering antisense to c-myc. In another embodiment, said method further comprises administering tumstatin. In one embodiment, said medium further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics.

One aspect of the present invention contemplates a method, comprising: a) providing: i) a medium comprising a compound selected from the group consisting of sirolimus, tacrolimus, analogs of sirolimus and pharmaceutically acceptable salts thereof, wherein said medium is selected from the group consisting of microparticles, gels, xerogels, hydrogels, bioadhesives, foams and combinations thereof; and ii) a patient, wherein said patient has a wound; and b) contacting said wound with said medium. In one embodiment, said wound is external. In another embodiment, said wound is internal. In one embodiment, said medium of step a) is housed in a device capable of delivering said medium to said wound. In one embodiment, said device delivers said medium by brushing. In one embodiment, said device delivers said medium by liquid administration. In one embodiment, said liquid administration comprises a liquid spray. In one embodiment, said liquid spray in the form of an aerosol. In one embodiment, said liquid administration comprises a pourable liquid. In another embodiment, said liquid administration comprises a squeezable liquid. In one embodiment, said device comprises a catheter. In one embodiment, said device is configured for endoscopic surgery. In one embodiment, said medium comprises a biocompatible material. In one embodiment, said medium comprises a biodegradable material. In one embodiment, said microparticles are selected from the group consisting of microspheres, microencapsulating particles, microcapsules and liposomes. In one embodiment, said medium is colored. In one embodiment, said medium further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said method further comprises administering antisense to c-myc. In another embodiment, said method further comprises administering tumstatin. In one embodiment, said medium further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics.

Another aspect of the present invention contemplates a method, comprising: a) providing: i) a composition, comprising a medium and a compound attached to said medium, said compound selected from the group consisting of sirolimus, tacrolimus, analogs of sirolimus and pharmaceutically acceptable salts thereof; and ii) a patient, wherein said patient has a surgical site; and b) contacting said surgical site with said composition. In one embodiment, said surgical site comprises a closed surgical site. In one embodiment, said composition of step a) is housed in a device comprising a reservoir, wherein said device is capable of delivering said composition to a surgical site. In one embodiment, said medium comprises a biocompatible material. In one embodiment, said medium comprises a biodegradable material. In one embodiment, said medium is selected from the group consisting of microparticles, gels, xerogels, hydrogels, bioadhesives, foams and combinations thereof. In another embodiment, said medium provides controlled release of said compound. In one embodiment, said microparticles are microencapsulating particles. In one embodiment, said microencapsulating particle is selected from the group consisting of microcapsules and liposomes. In one embodiment, said composition of step a) contacts said surgical site in the form of a spray. In one embodiment, said device delivers said composition by brushing. In one embodiment, said device delivers said medium by liquid administration. In one embodiment, said liquid administration comprises a liquid spray. In one embodiment, said liquid spray in the form of an aerosol. In one embodiment, said liquid administration comprises a pourable liquid. In another embodiment, said liquid administration comprises a squeezable liquid. In one embodiment, said device comprises a catheter. In one embodiment, said method further comprises observing said contacting of said surgical site with an endoscopic device. In another embodiment, said method further comprises observing said contacting of said surgical site with a fluoroscopic device. In one embodiment, medium is colored. In one embodiment, said medium further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said method further comprises administering antisense to c-myc. In another embodiment, said method further comprises administering tumstatin. In one embodiment, said medium further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics.

Another aspect of the present invention contemplates a method, comprising: a) providing: i) a composition, comprising a microparticle and a compound attached to said microparticle, said compound selected from the group consisting of sirolimus, tacrolimus analogs of sirolimus, and pharmaceutically acceptable salts thereof; and ii) a patient, wherein said patient has a surgical site; and b) contacting said surgical site with said composition. In one embodiment, said surgical site comprises a closed surgical site. In another embodiment, said surgical site comprises an open surgical site. In one embodiment, said composition of step a) is housed in a device comprising a reservoir, wherein said device is capable of delivering said composition to a surgical site. In one embodiment, said device delivers said composition by brushing. In one embodiment, said device delivers said composition by liquid administration. In one embodiment, said liquid administration comprises a liquid spray. In one embodiment, said liquid spray in the form of an aerosol. In one embodiment, said liquid administration comprises a pourable liquid. In another embodiment, said liquid administration comprises a squeezable liquid. In one embodiment, said device comprises a catheter. In one embodiment, said method further comprises observing said contacting of said surgical site with an endoscopic device. In another embodiment, said method further comprises observing said contacting of said surgical site with a fluoroscopic device. In one embodiment, said microparticle comprises a biocompatible material. In one embodiment, said microparticle comprises a biodegradable material. In one embodiment, said microparticle is a microsphere. In one embodiment, said microparticle is a microencapsulating particle. In one embodiment, said microencapsulating particle is selected from the group consisting of microcapsules and liposomes. In one embodiment, said microparticle is colored. In one embodiment, said microencapsulating particle further comprises a radio-opaque marker, wherein said marker is visualized by X-ray spectroscopy. In one embodiment, said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, said method further comprises administering antisense to c-myc. In another embodiment, said method further comprises administering tumstatin and antisense c-myc. In one embodiment, said medium further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics.

Another aspect of the present invention contemplates a method comprising: a) providing; i) a patient, wherein said patient has an open surgical site; ii) a biocompatible medium, wherein said medium is attached to a compound selected from the group consisting of sirolimus, tacrolimus, analogs of sirolimus and pharmaceutical acceptable salts thereof; and iii) a medical device containing said medium, wherein said medical device is capable of administering said compound to said surgical site; b) contacting said surgical site with said medium by administering said medium from said medical device; and c) reducing the formation of excess post-operative scar tissue and/or adhesions by pharmacological activity of said compound. In one embodiment, said medium is biodegradable. In one embodiment, said medium provides controlled release administration of sirolimus or analogs of sirolimus. In one embodiment, said medium comprises a microencapsulating particle. In another embodiment, said medium is selected from the group consisting of a gel, foam, dressing and bioadhesive. In one embodiment, said compound contacts said surgical site by liquid administration. In one embodiment, said contacting is selected from the group consisting of a spraying, brushing, wrapping and layering. In one embodiment, said microencapsulating particle is selected from the group consisting of microparticles, microspheres, microcapsules and liposomes. In one embodiment, said medium is comprised of a material selected from the group consisting of polylactide-polyglycolide polymers, lactide/glycolide copolymers, poly(lactide-co-glycolide) polymers (i.e., PLGA), hyaluronic acid, modified polysaccharides and any other well known substance that is known to be both biocompatible and biodegradable. In one embodiment said analog of sirolimus comprises a compound capable of binding to the mTOR protein selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said method further comprises administering antisense to c-myc. In another embodiment, said method further comprises administering tumstatin. In one embodiment, said medical device is selected from the group consisting of a self-contained spray container, a gas-propelled spray container, a spray catheter, a liquid-dispensing catheter, a brush, and a syringe. In one embodiment, said spray can comprises a single dose of said compound. In one embodiment, said spray can comprises a microencapsulating particle contacting said compound. In one embodiment, said medium is colored. In one embodiment, said medium further comprises a radio-opaque marker, wherein said marker is visualized by X-ray fluoroscopy. In one embodiment, said method further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics. In one embodiment, administration of said complementary pharmaceutical compound starts prior to exposure of said surgical site a surgical procedure. In another embodiment, administration of said complementary pharmaceutical compound continues for up to 6 months following exposure of said surgical site.

Another aspect of the present invention contemplates a method comprising: a) providing; i) a patient, wherein said patient has a closed surgical site; ii) a biocompatible medium, wherein said medium is attached to a compound selected from the group consisting of sirolimus, tacrolimus, analogs of sirolimus and pharmaceutically acceptable salts thereof; and iii) a medical device containing said medium, wherein said medical device is capable of, administering said medium to said surgical site; b) contacting said surgical site with said medium by administering said medium from said medical device; and c) reducing formation of excess post-operative scar tissue and/or adhesions by pharmacological activity of said compound. In one embodiment, said medium is biodegradable. In one embodiment, said method further comprises a step of, visualizing said surgical site with an endoscope to guide and verify said medium administration. In one embodiment, said analog of sirolimus comprises a compound capable of binding to the mTOR protein selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin. In one embodiment, said medium further comprises antisense to c-myc. In another embodiment, said medium further comprises tumstatin. In one embodiment, said medical device is selected from the group consisting of a catheter and said endoscope. In one embodiment, said catheter is capable of layering said medium. In one embodiment, said catheter is capable of spraying said medium. In one embodiment, said catheter is capable of liquid administration of said medium. In another embodiment, said catheter is capable of brushing said medium. In one embodiment, said catheter pours said medium. In another embodiment, said medium is selected from the group consisting of a microparticle, foam, gel, hydrogel, liquid spray and bioadhesive. In one embodiment, said method further comprises administering a supplemental pharmaceutical compound selected from the group consisting of antiinflammatory, corticosteriods, antithrombotics, antibiotics, antivirals, analgesics and anesthetics. In one embodiment, said method further comprises administering a complementary pharmaceutical compound selected from the group consisting of sirolimus, tacrolimus, analogs of sirolimus, antiinflammatory, corticosteriods, antithrombotics, antibiotics, antivirals, analgesics and anesthetics. In one embodiment, administration of said complementary pharmaceutical compound starts prior to exposure of said surgical site a surgical procedure. In another embodiment, administration of said complementary pharmaceutical compound continues for up to 6 months following exposure of said surgical site.

One aspect of the present invention contemplates a device, comprising: i) a reservoir containing a medium comprising sirolimus and analogs of sirolimus; ii) a fluid-driving element connected to said reservoir; iii) a channel having a first end and a second end, wherein said first end is connected to said reservoir; and iv) an extrusion port located at the second end of said channel, whereby said fluid-driving element causes said medium to extrude from said extrusion port.

One aspect of the present invention contemplates a device, said device comprising a reservoir comprising a medium comprising sirolimus and analogs of sirolimus and capable of delivering said medium to a surgical site. In one embodiment, said delivering is in the form of a spray. In one embodiment, said delivering is in the form of an aerosol. In one embodiment, said device comprises a catheter. In one embodiment, said device is an endoscope. In one embodiment, said endoscope is a laparoscope. One embodiment contemplates a surgical device wherein at least a portion of said device comprises an attached medium comprising sirolimus and analogs of sirolimus.

Another aspect of the present invention contemplates a device, said device comprising a reservoir comprising sirolimus and analogs of sirolimus and is capable of delivering said sirolimus and analogs of said sirolimus to a surgical site. In one embodiment, said delivering is in the form of a spray. In one embodiment, said delivering is in the form of an aerosol. In one embodiment, said device comprises a catheter. In one embodiment, said device comprises a laparoscopic device. In one embodiment, said device is a surgical device wherein at least a portion of said device is coated with sirolimus and analogs of sirolimus.

These and other embodiments and applications of this invention will become obvious to a person of ordinary skill in this art upon reading of the detailed description of this invention including the associated drawings.

### BRIEF DESCRIPTION OF THE DRAWINGS

FIG. 1 illustrates one embodiment of a liposome encapsulating a sirolimus molecule.

FIG. 2 illustrates one embodiment of a microsphere impregnated with a cytostatic anti-proliferative compound.

FIG. 3 illustrates one embodiment of a microsphere to which a cytostatic anti-proliferative compound is adhered to the surface.

FIG. 4 illustrates one embodiment of a microsphere comprising controlled release of sirolimus or an analog of sirolimus.

FIG. 5 illustrates one embodiment of a spray can to administer a sirolimus medium.

FIG. 6 shows one embodiment of a nebulizer tip attached to a syringe.

FIG. 7 illustrates by cross-section one embodiment of an endoscope shaft containing a endoscopic catheter delivering a medium.

FIG. 8 shows one embodiment of an endoscopic catheter for administration of media. Note: typical sizes are length=200 cm; diameter=2.5 mm and length of area 3 (w/ holes)=4 cm. Note: Female luer lock 81 that readily fits onto a syringe or other plunger device or spray can.

FIG. 9 shows one embodiment of a foam canister.

FIG. 10 shows one embodiment of surgical dressing.

FIG. 11 shows two exemplary embodiments of a side hole catheter.

FIG. 12 shows a close-up view of one embodiment of a slit port spray catheter tip.

FIG. 13 shows one embodiment of a bioadhesive applicator.

### DETAILED DESCRIPTION OF THE INVENTION

This invention is related to the field of tissue healing and reducing scar tissue formation. More specifically, this invention is related to the use of sirolimus and analogs of sirolimus (i.e., sirolimus and it's derivatives) to reduce post-surgical scar tissue formation. This invention also contemplates the use of compounds that are capable of binding to the mTOR protein to reduce and/or prevent scar tissue formation. The binding of compounds to the mTOR protein may be direct or indirect, competitive or non-competitive. Also contemplated are allosteric agonists or antagonists that may increase or decrease, respectively, the binding efficacy of a compound to the mTOR protein. Also contemplated by this invention are embodiments of antiproliferative, cytostatic compounds (i.e., sirolimus and analogs of sirolimus) which are believed to act primarily by interrupting the cell division cycle at the G0 or G1 phase in such a way that cell death does not occur.

Sirolimus and its derivatives is currently marketed as an antiproliferative, cytostatic drug in the liquid form for oral administration in 1-5 dosages per day of between 1-100 mg each. These disclosed oral mediums consist of conventional tablets, capsules, granules and powders. Guitard et al, Pharmaceutical Compositions. U.S. Pat. No. 6,197,781 (herein incorporated by reference).

Until recently, none of the clinical uses for the above liquid sirolimus compositions had been contemplated for the reduction of excess scar tissue. Sirolimus (i.e., rapamycin) is useful for treatment of post-surgical adhesions and scar tissue, wherein the drug is attached to a sheet of material and placed onto a damaged area. As the sirolimus is released from the sheet of material, it exerts it antiproliferative action. Fischell et al., Surgically Implanted Devices Having Reduced Scar Tissue Formation, U.S. Pat. No. 6,534,693 (2003).

One aspect of the present invention contemplates delivering sirolimus or other cytostatic agents to a surgical site or wound in a controlled release manner (i.e., ranging from 1 day to 6 months). In some embodiments described herein, a specific medium is contemplated comprising specific formulations of polymers that provide a controlled drug release capability where the polymers take the form of microparticles, gels, foams or liquids. In one embodiment, a local administration of a cytostatic compound is administered concurrently with a systemic administration of said cytostatic compound.

Another aspect of the present invention contemplates a variety of devices and methods to administer a medium comprising an attached compound. Preferably, these devices and methods include, but are not limited to, spray cans, reservoirs with plungers, delivery via a catheter for endoscopic procedures, premixed media, and media mixed at the time of administration.

Sirolimus and most analogs of sirolimus are known not to be readily soluble in aqueous solutions. A non-polar solvent or amphipathic material is usually required to generate a liquid solution (i.e., for example, olive oil). Otherwise, aqueous mixtures of sirolimus and analogs of sirolimus are limited to colloidal suspensions or dispersions. The present invention contemplates a method to improve the solubility of sirolimus. To this end, modified derivatives of sirolimus are contemplated in order to address this problem.

Soluble monoacyl and diacyl derivatives of sirolimus can be prepared according to known methods. Rakhit, U.S. Pat. No. 4,316,885 (herein incorporated by reference). These derivatives are used in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like. Furthermore, water soluble prodrugs of sirolimus may be used including, but not limited to, glycinates, propionates and pyrrolidinobutyrates. Stella et al., U.S. Pat. No. 4,650,803 (herein incorporated by reference).

Alternatively, aminoalkylation of sirolimus or analogs of sirolimus to create functional sirolimus derivatives is contemplated. Kingsbury et al. Synthesis Of Water-Soluble(Aminoalkyl)camptothecin Analogues: Inhibition Of Topoisomerase I And Antitumor Activity, J. Med. Chem. 34:98(1991). The Kingsbury et al. publication teaches the synthesis of several water-soluble analogs of camptothecin, by introduction of aminoalkyl groups into the camptothecin ring system. These derivatives retained their biological efficacy.

Alternatively, sirolimus or its analogs modified by bonding phenolic groups with diamines through a monocarbamate linkage is contemplated as having improved solubility. For example, it is known that the water solubility of camptotecin is improved by derivatives bonding to phenolic groups with diamines through a monocarbamate linkage. Sawada et al., Synthesis And Antitumor Activity Of 20(S)-Camptothecin Derivatives: Carbamate-Linked, Water Soluble Derivatives Of 7-Ethyl-10-hydroxycamptothecin, Chem. Pharm. Bull 39:1446 (1991).

It is known that beta-emitting radioisotopes placed onto a sheet of material reduce scar tissue formation. Although effective, the limited shelf life and safety issues associated with clinical use of radioisotopes make them less than ideal for routine use in the operating room or a doctor's office. Fischell et al., U.S. Pat. No. 5,795,286 (herein incorporated by reference).

Various means and methods to reduce scar tissue formation are disclosed in the art, but none utilizing the pharmacological activity of a cytostatic compound. For example, sheets of biodegradable mesh, gels, foams and barrier membranes of various materials, are commercially available or in clinical trials that are intended to reduce unwanted scar tissue growth and post-surgical adhesions. The mechanism of action of these barrier membranes is not pharmacological but involves a physical separation of the injured tissues, thereby preventing adherence.

### Actions of Sirolimus and Related Compounds

The present invention contemplates the administration of cytostatic, anti-proliferative compounds such as, but not limited to, sirolimus, tacrolimus (FK506) and any analog of sirolimus including, but not limited to everolimus (i.e., SDZ-RAD), CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin. In one embodiment, the present invention contemplates non-sirolimus compounds such as, but not limited to, antisense to c-myc (Resten-NG) and tumstatin.

Inhibition of mTOR

Cytostatic antiproliferative compounds, such as sirolimus (i.e. sirolimus) and its functional analogs are known to reduce cell proliferation. Originally discovered as an antifungal agent, the bacterial macrolide sirolimus is a potent immunosuppressant, a promising anti-cancer compound and an antiproliferative compound. Although it is not necessary to understand the mechanism of an invention, it is believed that sirolimus forms a complex with its cellular receptor, the FK506-binding protein (FKBP12), and inhibits the function of mammalian Target Of Rapamycin (mTOR). Current understanding indicates that by mediating amino acid sufficiency, mTOR governs signaling to translational regulation and other cellular functions by converging with the phosphatidylinositol 3-kinase pathway on downstream effectors. Recent findings have revealed a novel link between mitogenic signals and mTOR via the lipid second messenger phosphatidic acid that suggests mTOR may be involved in the integration of nutrient and mitogen signals. One hypothesis suggests that this possible interaction between phosphatidic and mTOR is inhibited by sirolimus binding. Chen et al, A Novel Pathway Regulating The Mammalian Target Of Sirolimus (mTOR) Signaling. Biochem Pharmacol. 64:1071-1077 (2002).

The binding of sirolimus, or sirolimus analogs, to the mTOR protein may be direct or indirect, or depend upon the binding of facilitating compounds, such as, allosteric agonists. Conversely, the binding of sirolimus or sirolimus analogs to the mTOR protein may depend on the binding of inhibiting compounds, such as, allosteric antagonists. Consequently, one of skill in the art would understand that the resultant change in mTOR protein activity due to the presence of sirolimus, or analogs of sirolimus, may not be solely dependent upon binding to sirolimus, or analogs of sirolimus.

Cell Cycle Interruption

Although it is not necessary to understand the mechanism of an invention, it is believed that the principal action of cytostatic antiproliferatives such as, sirolimus and analogs of sirolimus, is an interference with the progress of the cell cycle at the G0 or G1 phase. Other compounds capable of binding to the mTOR protein are also expected to decrease cellular proliferation and hence reduce the formation of excess scar tissue at a surgical or epidermal wound site. Compounds capable of binding to the mTOR protein may or may not have structure similarity to sirolimus or analogs of sirolimus nor may they have similar mTOR binding sites. Other cytostatic antiproliferatives that interfere with the G0 or G1 phase of the cell cycle are also contemplated within this invention to effectively reduce scar tissue when properly dispensed to a surgical or other injury site.

Sirolimus and its analogs impact a variety of cell types. In the case of preventing vascular hyperplasia following angioplasty it is believed that the dominant mechanism of sirolimus released at the site of a vascular stent is to inhibit growth factor and cytokine mediated smooth muscle cell proliferation at the G1 phase of the cell cycle. In its application as an anti-rejection drug, sirolimus is administered systemically to prevent T-cell proliferation and differentiation. Moses, J. W., Brachytherapy And Drug Eluting Stents, J Invasive Cardiology, 15:30B-33B (2003).

Glib-1 oncongeny expression occurs in both scar tissue and keloids, wherein keloids express greater hyperproliferative characteristics, and glib-1 expression, than ordinary scar tissue. Since sirolimus is known to inhibit glib-1 oncogeny expression it is expected that sirolimus also inhibits glib-1 expression in keloids. Kim et al., Are Keloid Really “Glib-loids”: High-Level Expression Of Glib-1 Oncogeny In Keloid. J. Am Acad Dermatol 45(5):707-711 (2001). In one embodiment, the present invention contemplates a reduction in keloid formation following the administration of sirolimus or analogs of sirolimus.

### Actions of Non-Sirolumus Related Compounds

Cytotoxic/Antiproliferative Compounds

Other cytotoxic compounds (i.e., taxol and other anticancer compounds), may or may not bind to the mTOR protein, and have anti-proliferative effects, but are typically cytotoxic. These cytotoxic compounds interfere with proliferation in part by interfering with successful cell division in stage G2 or M, resulting in cell death. Because the by-products of cell death are in and of themselves inflammatory and stimulative, it is believed that stopping cell proliferation with a cytostatic effect rather than a cytotoxic effect is preferred. Indeed, in drug coated stent trials, arteries treated with stents coated with a cytostatic drug (sirolimus) showed less neointimal tissue growth than vessels treated with stents coated with a cytotoxic drug (paclitaxel). Grube et al., Taxus I: Six And Twelve Month Results From A Randomized, Double Blind Trial On Slow Release Paclitaxel Eluting Stent For De Novo Coronary Lesions. Circulation 107:38-42 (2003); and Morice et al., A Randomized Comparison Of Sirolimus-Eluting Agent With A Standard Stent For Coronary Revascularization. N Engl J Med 346:1773-1780 (2002).

The present invention also contemplates cytotoxic anti-proliferative non-sirolimus compounds including, but not limited to, anticancer compounds such as taxol, actinomycin-D, alkeran, cytoxan, leukeran, cis-platinum, BiCNU, adriamycin, doxorubicin, cerubidine, idamycin, mithracin, mutamycin, fluorouracil, methotrexate, thioguanine, toxotere, etoposide, vincristine, irinotecan, hycamptin, matulane, vumon, hexalin, hydroxyurea, gemzar, oncovin and etophophos. Preferably, cytotoxic antiproliferative non-sirolimus compounds are used in combination with sirolimus, tacrolimus and analogs of sirolimus. Alternatively, cytotoxic antiproliferative non-sirolimus compounds may also be used alone.

Non-Sirolimus mTOR Binding

One embodiment of the present invention contemplates the reduction of excess scarring by compounds capable of inhibiting the mTOR protein. One exemplary compound is tumstatin, a 28-kilodalton fragment of type IV collagen that displays both anti-angiogenic and proapoptotic activity. Tumstatin is known to function as an endothelial cell-specific inhibitor of protein synthesis, however, there is no speculation in the art regarding any ability to reduce excess scar tissue. Although it is not necessary to understand an invention, it is believed that tumstatin acts through αVβ3integrin, inhibits focal adhesion kinase, phosphatidylinositol 3-kinase, protein kinase B, mTOR, and prevention of the dissociation of eukaryotic initiation factor 4E protein (eIF4E) from 4E-binding protein 1.

### Current Clinical Applications of Sirolimus

Although there are several known uses of sirolimus, none of them include a combination of sirolimus and a medium where the medium is in the form of a microsphere, gel, liquid, bioadhesive or foam. Also none of the known uses contemplate using such a composition to prevent excess scar tissue growth following injury. In such a form, the sirolimus can be adminsitered to the wound site easily, without missing any portions of the affected tissue.

Scar Tissue Reduction

Sirolimus (i.e., rapamycin) attached to a sheet of material is known as a useful treatment of post-surgical treatment of adhesions and scar tissue. Fischell et al., Surgically Implanted Devices Having Reduced Scar Tissue Formation, U.S. Pat. No. 6,534,693 (2003). Scarring, as used herein, also contemplates the narrowing of any neurological, vascular, ductal/tubal (e.g., for example, pancreatic, biliary or fallopian) space in the body secondary to injury from, for example, implants, trauma, surgery or system and local disease/infections. In one embodiment, the present invention contemplates the administration of sirolimus, tacrolimus and analogs of sirolimus to reduce scarring in compositions comprising a medium including, but not limited to, a foam, gel, bioadhesive that may or may not be attached to a dressing or medical device. Further, the present invention contemplates the long term administration of sirolimus in the prevention of scar tissue formation by compositions that provide controlled release of sirolimus or related compounds.

Transplantations

Sirolimus (i.e., Rapamune®: Wyeth, Madison, N.J.) is known as an immunosuppressant effective for long-term immunosuppressive therapy in renal transplantation. Observations indicate that sirolimus operates synergistically with cyclosporin A (CsA). For example, in blinded dose-controlled trials, the rates of acute rejection episodes within 12 months following administration of 2 or 5 mg/day sirolimus in combination with CsA and steroids were reduced to 19 and 14%, respectively. It is speculated that sirolimus acts to retard proliferation of vascular smooth muscle cells, an important component of the immuno-obliterative processes associated with chronic rejection. Kahan, Sirolimus: A Comprehensive Review. Expert Opin Pharmacother 2:1903-17 (2001).

The administration of mTOR inhibitors (i.e., sirolimus) are known to result in improved outcomes for renal transplant recipients by decreasing the risk of rejection and by increasing the function and lifespan of the allograft. Gourishankar et al., New Developments In Immunosuppressive Therapy In Renal Transplantation. Expert Opin Biol Ther 2:483-501 (2002)

Tacrolimus and sirolimus are two immunosuppressive compounds considered as optimal immunosuppressive strategies for pancreas transplantation. Specifically, the application of these compounds have contributed to substantially lower rates of allograft rejection and improved graft survival. Odorico et al., Technical And Immunosuppressive Advances In Transplantation For Insulin-Dependent Diabetes Mellitus. World J Surg 26:194-211 (2002). Similar effects on renal transplantation success has been reported following the administration of an analog of sirolimus; SDZ RAD (everolimus, Certican®). Nashan, Early Clinical Experience With A Novel Sirolimus Derivative. Ther Drug Monit 24:53-8 (2002).

Vascular Stents

Sirolimus is known as a coating for intraluminal vascular medical devices and methods of treating intimal hyperplasia, constrictive vascular remodeling and resultant vascular scarring and injury-induced vascular inflammation. Falotico et al., Compound/Compound Delivery Systems For The Prevention And Treatment Of Vascular Disease. Published U.S. patent application Ser. No. 2002/0007214 A1, Published U.S. patent application Ser. No. 2002/0007215 A1, U.S. patent application Ser. No. 2001/0005206 A1, U.S. patent application Ser. No. 2001/007213 A1, U.S. patent application Ser. No. 2001/0029351 A1; and Morris et al., Method Of Treating Hyperproliferative Vascular Diseases. U.S. Pat. No. 5,665,728. These conditions are generally referred to as hyperproliferative vascular disease and may be caused by vascular catheterization, vascular scraping, percutaneous transluminal/coronary angioplasty, vascular surgery, vascular endothelial proliferation, intimal hyperplasia, foreign body endothelial proliferation, and obstructive proliferation/hyperplasia including specific conditions such as, but not limited to, fibroblastic, endothelial or intimal.

Vascular stents have been coated with sirolimus (i.e., sirolimus), actinomycin-D or taxol to reduce cellular proliferation and restenosis following angioplasty or recanalization of injured arteries. However, these compositions have never been used for reducing cellular proliferation at the site of a surgical procedure. Hossainy et al., Process For Coating Stents. U.S. Pat. No. 6,153,252.

Sirolimus has been demonstrated to inhibit smooth muscle cell (SMC) proliferation and migration in vitro and to reduce in vivo neointima formation by blocking the cell cycle before the G1-S transition. Further, sirolimus drug-eluting stents eliminate restenosis after stent implantation. Paclitaxel (Taxol: a microtubule-stabilizing agent) has a similar antiproliferative effect. Paclitaxel, however, is believed to act by inhibiting spindle formation necessary for cell division. Chieffo et al., Drug-Eluting Stents. Minerva Cardioangiol 50:419-29 (2002).

Keloids

Related to scars are lesions known as keloids. Keloids arise from sites of previous trauma. Keloids are a considerable source of morbidity because of continued growth, pruritus, and physical appearance. Clinically, keloids are distinguished from scars in that keloids continue to grow over the borders of the original injury. It has been observed that both sirolimus and tacrolimus (i.e., FK506; an antiproliferative) effectively treat keloids. Kim et al., Are Keloid Really “Glib-loids”: High-Level Expression Of Glib-1 Oncogeny In Keloid. J. Am Acad Dermatol 45(5):707-711 (2001).

TNF-β Ligand

Also, a combination of a sirolimus derivative with a tissue growth factor-beta ligand is known to prevent the formation of ocular scar tissue and/or promote the proliferation of connective tissue or soft tissue for wound healing. Donahoe et al., Methods And Compositions For Enhancing Cellular Response To TGF-β Ligands. U.S. Pat. No. 5,912,224.

Clearly, still lacking in the art is any contemplation that sirolimus and analogs of sirolimus may be effective either during, or after, a surgical procedure to reduce or prevent the formation of scar tissue on any living tissue.

### PREFERRED EMBODIMENTS OF THE PRESENT INVENTION

Excess scar tissue production is a known morbidity consequence of healing from a number of types of wounds. Examples include, but are not limited to, hypertrophic burn scars, surgical adhesions (i.e., for example, abdominal, vascular, spinal, neurological, thoracic and cardiac), capsular contracture following breast implant surgery and excess scarring following eye surgery and ear surgery.

The delivery of specific compounds contemplated by this invention to a surgical site or wound include, but are not limited to, microparticles, gels, hydrogels, foams, bioadhesives, liquids, xerogels or surgical dressings. Particularly, these media are produced in various embodiments providing a controlled release of a compound such as sirolimus.

### Clinical Applications

Burns

Burn injuries are well known for the development of scar tissue during the healing process. Sirolimus and analogs of sirolimus are contemplated by the present invention to be applied by any one of the compositions and methods described herein to facilitate the healing and reduction and/or prevention of scar tissue and adhesions of a burn wound.

The clinical management of burn-induced hypertrophic scarring has focused primarily on the application of pressure since the early 1970s. Although the exact mechanism of action is unknown, pressure appears clinically to enhance the scar maturation process. Bandages that can be wrapped and unwrapped or are made of a soft material are used in early scar management. Custom-made pressure garments generally are used for definitive scar management and inserts are placed in concavities to aid in compression. Staley et al., Use Of Pressure To Treat Hypertrophic Burn Scars. Adv Wound Care 10:44-46 (1997). However, it is clear that these approaches, while helpful, still allow the development of serious and debilitating scarring.

The further development of effective topical chemotherapy, reintroduction of burn wound excision, and the use of biologic dressings have significantly decreased the incidence of invasive burn wound infection and have contributed to the improvement in the survival over the past four decades. The currently available skin substitutes, however, are imperfect and research endeavors are essential to continue to develop a nonantigenic and disease-free physiologically effective tissue (i.e., synthetic skin). This approach will eventually improve wound closure, reduce scar formation thereby reducing the need for reconstructive surgery. Greenfield et al., Advances In Burn Wound Care. Crit Care Nurs Clin North Am 8:203-15 (1996).

The advent of specific antiproliferative drugs (i.e., sirolimus and analogs of sirolimus) that reduce scarring in burn patients will provide an enormous benefit to burn patients. Specifically, by controlling the overgrowth of scar tissue, the normal healing process will be allowed to predominate. As such, the need for post-burn healing medical treatments to provide cosmetic, and clinical, treatment for burn scars will be minimized. The present invention specifically contemplates a method to reduce scars comprising: a) providing; i) sirolimus or an analog of sirolimus or other cytostatic antiproliferative, ii) a burn patient; and b) administering said sirolimus or analog of sirolimus to said burn patient under conditions such that scarring is reduced. Preferably, said sirolimus, analog of sirolimus or other active compound is delivered locally at a burn site on the skin, either with or without a systemic concurrent administration of said active compound, such as sirolimus.

Pericarditis

Pericarditis is an inflammation and swelling of the pericardium (i.e., the sac-like covering of the heart), which can occur in the days or weeks following a heart attack.

Examples of clinical conditions involving pericarditis include, but are not limited to, Dressler's syndrome, post-myocardial infarction, post-cardiac injury, and postcardiotomy.

Pericarditis may occur within 2 to 5 days after a heart attack (i.e., for example, an acute myocardial infarction), or it may occur as much as 11 weeks subsequent to such an attack and may involve repeated episodes of the symptoms. Pericarditis may also result from open heart surgery, stab wounds to the heart and blunt chest trauma.

Pericarditis occurring shortly after a heart attack is caused by the inflammatory response to blood in the pericardial sac or by the presence of dead or severely damaged tissue in the heart muscle. During the period of inflammation, the immune system sometimes healthy cells by mistake. Pain occurs when the inflamed pericardium rubs on the heart.

Early pericarditis complicates 7% to 10% of heart attacks. Dressler's syndrome is seen in only 1% of patients after heart attack. Risks include previous heart attack, open heart surgery or chest trauma.

In one embodiment, the present invention contemplates a method to reduce scars and inflammation following heart surgery wherein sirolimus, tacrolimus, analogs of sirolimus or another cytostatic antiproliferative drug are administered to a patient exhibiting symptoms of pericarditis. In another embodiment, the present invention contemplates a method to reduce scars and inflammation wherein sirolimus, tacrolimus, analogs of sirolimus or another cytostatic antiproliferative drug are administered to a patient undergoing heart surgery so as to prevent pericarditis.

Surgical Adhesions

Postsurgical adhesions are fibrous scar tissue formations, or fibrin matrices, that form between tissues or organs following injury associated with surgical procedures. Such injuries include ischemia, foreign body reaction, hemorrhage, abrasion, incision, and infection-related inflammation. In the U.S., the annual cost of removing lower abdominal adhesions is estimated to be more than $2 billion in inpatient treatment charges. Adhesions also develop following cardiac, spinal, neurological, pleural and other thoracic surgery. In one embodiment, the present invention contemplates a reduction in pleural adhesions following lung surgery. In another embodiment, the present invention contemplates a reduction in cardiac adhesions following cardiac surgery.

Postsurgical damage sites form adhesions in tissues or organs that normally remain separate, but instead, join together by fibrin matrices within the first few days following surgery. Under normal circumstances, most fibrin matrices between organs degrade during the healing process. When fibrin matrices fail to degrade, permanent adhesions are formed, linking tissues and/or organs together. Such unwanted adhesion formation following gynecologic or general abdominal surgeries can lead to a variety of complications, including pain, infertility and bowel obstruction.

Adhesions are recognized as serious sequelae in patients undergoing gynecologic and general abdominal surgical procedures. For example, the presence of adhesions between structures such as the fallopian tubes, ovaries and uterus following surgery is a major cause of pain and infertility.

Abdominal adhesions are the predominant cause of small-bowel obstruction, accounting for 54% to 74% of cases. Moreover, approximately 80% to 90% of abdominal adhesions result from surgery.

Pelvic adhesions occur in 55% to 100% of fertility-enhancing procedures as determined by second-look laparoscopy performed in a number of large, multicenter studies.

In an attempt to reduce the tissue trauma and thus recovery time, special microsurgical medical procedures have been developed that minimize tissue handling. However, even when these techniques are followed, postoperative adhesions can occur in the majority of patients in certain surgical procedures. Therefore, it is generally believed that the best approach to minimizing postsurgical adhesion formation is through the use of special microsurgical techniques in combination with anti-adhesion protocols.

The reduction of post-surgical adhesions following liquid spray applications of fluorocarbons to open surgical sites is known. These fluorocarbons act by coating the tissue and reducing surface tension, thus preventing adherence of the coated tissues when brought into close proximity. Niazi, S., Use Of Fluorocarbons For The Prevention Of Surgical Adhesions. U.S. Pat. No. 6,235,796.

Another method to reduce surgical tissue adhesion by physical barrier means utilizes a dual chamber spray can or bottle that mixes two polymer solutions at the nozzle. This mixing initiates a nucleophilic-electrophilic crosslinking reaction and generates a solidified polymer matrix. Either polymer mixture is also capable of delivering growth factors to a surgical site as part of a bioadhesive polymer matrix. The polymer matrix prevents post-surgical adhesion formation via the tissue surface coating and is capable of removing scar tissue. Synthetic polymers of collagen or hyaluronic acid are specifically contemplated, and natural proteins may be added to improve the bioadhesive properties of the matrix. Rhee et al., Method Of Using Crosslinked Polymer Compositions In Tissue Treatment Applications. U.S. Pat. No. 6,116,139 (herein incorporated by reference).

Alternatively, it is known that post-surgical adhesions are prevented or reduced by the administration of another type of barrier, a thermally gelling polymer. Gelation of a thermal gel during its administration is determined by its phase transition temperature. It is known in the art that the thermal gel phase transition temperature may be modified by mixing a modifier polymer (i.e., cellulose esters or Carbopol®) with a constitutive polymer (i.e., polyoxyalkene copolymer). Flore et al., Methods And Compositions For The Delivery Of Pharmaceutical Agents And/Or The Prevention Of Adhesions. U.S. Pat. No. 6,280,745 (herein incorporated by reference). The '745 patent explains that prevention of post-surgical scarring and adhesions is due the actual physical presence of the gel (i.e., acting as an artificial barrier to growth), rather than due to the pharmacological action of any compound delivered with the hydrogel.

The present invention contemplates the administration of a medium comprising a cytostatic and antiproliferative compound (i.e., such as, for example, sirolimus, tacrolimus and/or analogs of sirolimus) to a surgical site or other area of tissue injury that, by pharmacological action, prevents or reduces the formation of scar tissue and post-surgical adhesions. In a preferred embodiment, the medium comprising the cytostatic or antiproliferative compound is easily administered to the surgical field via liquid administration techniques, via a thermally gelling polymer, via a bioadhesive or via microparticles.

External Vascular Scarring

The present invention contemplates a medium comprising a cytostatic and antiproliferative compound (i.e., sirolimus, tacrolimus and analogs of sirolimus) applied to an external vascular site. In one embodiment, the compound reduces or prevents the formation of scar tissue or tissue adhesions.

The advent of permanent hemodialysis access has made possible the use of chronic hemodialysis in patients with end-stage renal disease. Although autogenous arteriovenous fistulae remain the conduit of choice, their construction is not always feasible. Prosthetic grafts made of polytetrafluoroethylene (PTFE) are typically the second-line choice for hemoaccess. However, these grafts suffer from decreased rates of patency and an increased number of complications. Anderson et al., Polytetrafluoroethylene Hemoaccess Site Infections, American Society for Artificial Internal Organs Journal, 46(6):S18-21 (2000). In one embodiment, the present invention contemplates the administration of a medium comprising sirolimus, tacrolimus or an analog of sirolimus to a patient having PTFE graft complication. In one embodiment, the medium is sprayed onto the PTFE graft. In another embodiment, the medium is attached to a surgical wrap that encircles the PTFE graft. In one embodiment, the medium is attached to a surgical sleeve (i.e., a bandage or mesh that is tubular in nature) that is placed onto the exterior surface of the vasculature during the PTFE graft procedure.

Ear Scarring

One aspect of the present invention contemplates a method of applying sirolimus, tacrolimus and analogs of sirolimus in and around the ear to prevent progressive inner ear deterioration (i.e., cholesteatoma). It is known that process of scar formation within the ear, including the ear drum, is very similar to other tissues. The epithelial pathogenesis of acquired cholesteatoma appears to have three prerequisites: (1) the unique anatomical situation at the ear-drum (two different epithelial layers close together); (2) chronic destruction of the submucosal tissue in the middle ear (infection, inflammation); and (3) wound healing (i.e., a proliferation phase). Destruction of the submucosal space by middle ear infection and cell necrosis starts the wound healing cascade. In wound healing, generally the connective tissue fibroblasts and macrophages play a pivotal role. Cytokines are thought to promote the re-epithelization of the mucosal defect and scar tissue development act upon the intact squamous cell layer of the outer surface of the ear-drum at the same time. Thereby a proliferation of the undamaged epithelial layer is induced. Cholesteatoma matrix is always surrounded by a layer of connective tissue, the perimatrix. Persistence of the inflammation causes permanent wound healing in the perimatrix, proliferation of the fibroblasts (granulation tissue) and proliferation of the epithelium (matrix). It is speculated that by virtue of wound healing cytokines of fibroblasts and macrophages are the driving forces of cholesteatoma origin, growth and bone destruction. Milewski C., Role Of Perimatrix Fibroblasts In Development Of Acquired Middle Ear Cholesteatoma. A Hypothesis. HNO 46:494-501 (1998).

The present invention contemplates the administration of a medium comprising a cytostatic and antiproliferative compound including, but not limited to, sirolimus, tacrolimus and/or analogs of sirolimus, to the ear so that, by pharmacological action, such excess scar tissue is prevented or reduced.

Eye Scarring

One aspect of the present invention contemplates a method of applying sirolimus, tacrolimus, and analogs of sirolimus to eye tissues following or during surgery or trauma. Various conditions of the eye are known to be associated with corneal scarring and fibroblast proliferation, including ocular coagulation and burns, mechanical and chemical injury, ocular infections such as kerato-conjunctivitis, and other ocular conditions. Some of these conditions are known to arise post-operatively after surgical treatment of other ocular conditions. This undesirable tissue growth is easily neovascularized and therefore becomes permanently established and irrigated. Tissue scarring or fibroblast proliferation is a condition which is difficult to treat. Presently, it is treated by subjecting the ocular area to further surgery or by using steroids, topically or by injection. However, steroids do increase side effects such as infection, cataract and glaucoma. Other non-steroidal agents like indomethcin have very little anti-scarring effects. (Williamson J. et al., British J. of Ophthalmology 53:361 (1969); Babel, J., Histologie Der Crtisonkatarakt, p.327. Bergmann, Munich (1973)).

It is known in the art that corneal scarring, neovascularization or fibroblast proliferation maybe reduced by the application of a human leukocyte elastase (HLE) inhibitory agents (i.e., carbamates substituted by oligopeptides). Digenis et al. Methods Of Treating Eye Conditions With Human Leukocyte Elastase (HLE) Inhibitory Agents. U.S. Pat. No. 5,922,319. Elastases (human leukocyte elastase and cathepsin G), appear to be responsible for some chronic tissue destruction associated with inflammation, arthritis and emphysema. Therefore, the actions of elastase inhibitors do not involve the mTOR protein in regards to their antiproliferative effects relative to the reduction of scar tissue formation.

Progressive scarring may result in blindness, especially in cases where the retina is involved. The most common cause of failure of retinal reattachment surgery is formation of fibrocellular contractile membranes on both surfaces of the neuroretina. This intraocular fibrosis, known as proliferative vitreoretinopathy, results in a blinding tractional retinal detachment because of the contractile nature of the membrane. Contractility is a cell-mediated event that is thought to be dependent on locomotion and adhesion to the extracellular matrix. Sheridan et al., Matrix Metalloproteinases: A Role In The Contraction Of Vitreo-Retinal Scar Tissue. Am J Pathol 159:1555-66 (2001).

Corneal wound healing frequently leads to the formation of opaque scar tissue. Stromal fibroblastic cells of injured corneas express collagen IV and contribute to the formation of a basal lamina-like structure. Normally, stromal collagenous matrix organizes in orthogonal lamellae during corneal development, whereas that of an alkali-burned cornea, is known to develop in a disorganized manner. Enhanced expression of collagen IV by the fibroblastic cells in the stroma of injured corneas is consistent with the notion that they may contribute to the formation of basal lamina-like structures in injured corneas. Ishizaki et al., Stromal Fibroblasts Are Associated With Collagen IV In Scar Tissues Of Alkali-Burned And Lacerated Corneas. Curr Eye Res 16:339-48 (1997).

Medical Devices

One aspect of the present invention contemplates a method for applying sirolimus, tacrolimus and analogs of sirolimus to reduce scar tissue formation and adhesions following the placement of medical device implants.

Excess scar tissue formation and inflammation around direct medical implants are of particular concern. For example, the permanent placement of a percutaneous functional implant that protrudes through the skin for prolonged periods of time has not yet become a reality. Efforts towards eventual success must be directed toward a variety of failure mechanisms. For example, these mechanisms may be either extrinsic or intrinsic that cause shearing and tearing at the skin-implant interface. Extrinsic forces are defined as those forces applied either to the skin or the implant by the external environment. Intrinsic forces are those that have to do directly or indirectly with the body's growth and cell maturation, such as the retraction of maturing scar tissue and the surface migration of squamous epithelium. An intact skin-implant interface is important to attain in order to provide a seal against microbial invasion. The skin must remain intact, since a suppurative wound makes the implant's removal mandatory. Hall et al., Some Factors That Influence Prolonged Interfacial Continuity. J Biomed Mater Res 18:383-93 (1984).

Implants for reconstructive or cosmetic surgery, such as breast implants, also have problems with excess scar tissue formation. Breast implants are known to develop surrounding scar capsules that may harden and contract, resulting in discomfort, weakening of the shell with rupture, asymmetry, and patient dissatisfaction. This phenomenon is known to occur in as many as 70 percent of implanted patients over time. Most complications are due to late leaks, infection, and capsular contracture. Ersek et al., Textured Surface, Nonsilicone Gel Breast Implants: Four Years' Clinical Outcome. Plast Reconstr Surg 100:1729-39 (1997).

Glaucoma implants are also suspected to fail due to scar formation. Glaucoma implants are designed to increase fluid outflow from the eye in order to decrease intraocular pressure and prevent damage to the optic nerve. The implant consists of a silicone tube that is inserted into the anterior chamber at one end and is attached at the other end to a silicone plate that is sutured to the outside of the globe beneath the conjunctiva. The glaucoma “implant” becomes a “drain” over the first 3 to 6 postoperative weeks as the silicone plate is enclosed by a fibrous capsule that allows a space to form into which fluid can drain and from which fluid can be absorbed by the surrounding tissues. Ideally, the size and thickness of the capsule (i.e., the filtering bleb) that surrounds the plate is such that the amount of fluid that passes through the capsule is identical to the amount of fluid produced by the eye at an intraocular pressure of 8 to 14 mmHg. The most common long-term complication of these implants is failure of the filtering bleb 2 to 4 years after surgery due to the formation of a thick fibrous capsule around the device. Micromovement of the smooth drainage plate against the scleral surface may be integral to the mechanism of glaucoma implant failure by stimulating low-level activation of the wound healing response, increased collagen scar formation, and increased fibrous capsule thickness. Jacob et al. Biocompatibility Response To Modified Baerveldt Glaucoma Drains. J Biomed Mater Res 43:99-107 (1998).

Another aspect of the present invention contemplates coating a medical device with a medium comprising sirolimus, tacrolimus or an analog of sirolimus. A “coating”, as used herein, refers to any compound that is attached to a medical device. For example, such attachment includes, but is not limited to, surface adsorption, impregnation into the material of manufacture, covalent or ionic bonding and simple friction adherence to the surface of the medical device.

Sirolimus or analogs of sirolimus may be attached to a medical device in a number of ways and utilizing any number of biocompatible materials (i.e., polymers). Different polymers containing sirolimus are utilized for different medical devices. For example, a ethylene-co-vinylacetate and polybutylmethacrylate polymer is utilized with stainless steel. Falotico et al., U.S. patent application, 20020016625. Other polymers may be utilized more effectively with medical devices formed from other materials, including materials that exhibit superelastic properties such as alloys of nickel and titanium. In one embodiment, a compound such as, but not limited to, sirolimus, tacrolimus or analogs of sirolimus are directly incorporated into a polymeric matrix and sprayed onto the outer surface of a catheter such that the polymeric spray becomes attached to said catheter. In another embodiment, said compound will then elute from the polymeric matrix over time and enter the surrounding tissue. In one embodiment, said compound is expected to remain attached on the catheter for at least one day up to approximately six months.

In one embodiment, the present invention contemplates a sirolimus hydrogel polymer coating on a stainless steel medical device (i.e., for example, a permanent implant). Preferably, a stainless steel implant is brush coated with a styrene acrylic aqueous dispersion polymer (55% solids) and dried for 30 minutes at 85° C. Next, this polymer surface is overcoated with a controlled release hydrogel composition consisting of:

| | | |
|---|---|---|
| Polyvinyl pyrrolidone (PVP) | 9.4 | gm |
| Ethanol | 136.1 | gm |
| Butyrolactone | 30.6 | gm |
| 0.0625% nitrocellulose in cyclohexanone | 3.8 | gm |
| Sirolimus (dissolved in olive oil) | 10 | mg/ml |

The coating is then dried for 25 hours at 85° C. prior to use. It is not intended that the present invention be limited by the above sirolimus concentration. One skilled in the art should realize that that various concentrations of sirolimus may be used such as, but not limited to, 1.0-10 mg/ml, preferably 0.1-5 mg/ml, and more preferably 0.001-1 mg/ml.

In another embodiment, a multiple layering of non-erodible polymers may be utilized in conjunction with sirolimus. Preferably, the polymeric matrix comprises two layers; a inner base layer comprising a first polymer and the incorporated sirolimus and an outer second polymer layer acting as a diffusion barrier to prevent the sirolimus from eluting too quickly and entering the surrounding tissues. In one embodiment, the thickness of the outer layer or top coat determines the rate at which the sirolimus elutes from the matrix. Preferably, the total thickness of the polymeric matrix is in the range from about 1 micron to about 20 microns or greater. Another embodiment of the present invention contemplates spraying or dipping a polymer/sirolimus mixture onto a catheter.

Intraluminal Narrowing

The formation of excess scar tissue and resultant intraluminal narrowing in bodily lumens is a well known phenomenon following illness, injurious trauma, implants or surgery that involves bodily organs. The mechanisms for such narrowing include fibroblastic, endothelial and intimal excess proliferation or hyperplasia. Perhaps the most well-known condition is that of restenosis, which is a condition of a narrowing of the vascular lumen following systemic or local hyperproliferative vascular disease, or as a complication of vascular surgery, injurious trauma or implantation of a medical device. Other examples of excess luminal narrowing occur following ductal/tubal surgery, including, but not limited to, pancreatic, biliary and fallopian tube surgery.

Although it is not intended to limit the present invention, it is believed that the following example regarding arteriovenous fistula blockage provides an adequate teaching.

Vascular access complications include, but are not limited to, arteriovenous fistulae which is a major problem in hemodialysis patients. The most common complication is progressive stenosis at the anastomotic site. In most cases, this stenosis occurs at the venous anastomotic site.

Vascular access is governed by the DOQI (Dialysis Outcome Quality Initiatives). In early 2000, the National Kidney Foundation (NKF) announced it is expanding the scope of DOQI study to include “all phases of kidney disease and dysfunction and their monitoring and management.” DOQI has developed and published clinical practice guidelines in four areas—hemodialysis, peritoneal dialysis, anemia, vascular access and nutrition.

Thus, according to DOQI, patients requiring vascular access are treated with the following progression of dialysis vascular access grafts as they fail: i) a Cimino graft; which is a lower forearm radial artery/cephalic vein A-V fistula (i.e., a native graft); ii) an upper arm native fistula; connecting the brachial artery to either the cephalic or basilic vein; and iii) an upper arm PTFE Loop; connecting the brachial artery to the median antecubical vein.

The primary failure issues related to graft technology are that: i) even though 70% Cimino grafts are suitable for use 50% fail over the first ten years and 30% generate thromboses or fail to mature (i.e., undergo endothelization and healing); ii) a condition known as “steal” develops that is characterized by a high blood flow rate through the graft (i.e., 300-500 ml/min) resulting in a lack of blood flow to the hand and lower arm and iii) PTFE grafts typically develop initimal thickening at the venous anatomotic site.

One approach to remedy these problems is to apply a perivascular endothelial cell implant to inhibit intimal thickening observed following chronic arteriovenous anastomoses. Nugent et al., Perivascular Endothelial Implants Inhibit Intimal Hyperplasia In A Model Of Arteriovenous Fistulae: A Safety And Efficacy Study In The Pig. J Vasc Res 39(6):524-33 (2002). In one embodiment, the present invention contemplates a method to reduce scar tissue formation following an arteriovenous anastomosis in a dialysis patient. In another embodiment, said patient has end stage renal disease. In another embodiment, the patient has an artificial graft.

Another aspect of the present invention contemplates treatment of vascular complications following coronary or peripheral bypass graft surgeries. It is well known that arterial grafts have a higher success rate than autologous venous grafts. However, venous grafts remain preferred as they are easier to harvest and insert and far more available. A major disadvantage to using venous grafts lies in the fact that 10%-18% fail within 1-6 months following surgery, due predominately to exaggerated intimal hyperplasia. Hyperplasia may be accompanied by neointimal thickening and atherosclerotic plaques. Improvement in vein graft patency, therefore, remains a long felt need in this area of vascular surgery.

The present invention contemplates a method to improve the patency of vascular grafts by administration of a medium comprising sirolimus, tacrolimus and analogs of sirolimus following any surgical manipulation (i.e, for example, suturing) that results in a direct trauma to the endothelium and smooth muscle cells of the vasculature. In one embodiment, the administration of said medium reduces anastomotic and vein graft intimal hyperplasia believed caused by an intrinsic adaptive response of the medial smooth muscle cells.

Transplantations

One aspect of the present invention contemplates a medium comprising a cytostatic and antiproliferative compound (i.e., for example, sirolimus, tacrolimus and analogs of sirolimus) administered to a patient during and after an organ transplant. In one embodiment, a method results in the prevention or reduction of post-transplantation scarring. It is well known in the art that sirolimus and related compounds are effective in reducing the graftversus-host rejection cascade. This invention, however, proposes a novel use in regards to prevention of scarring for sirolimus in this clinical setting.

### Drug Delivery Systems

The present invention contemplates several drug delivery systems that provide for roughly uniform distribution, have controllable rates of release and may be administered to either an open or closed surgical site. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One aspect of the present invention contemplates a medical device comprising several components including, but not limited to, a reservoir comprising sirolimus, tacrolimus or an analog of sirolimus, a catheter, a sprayer or a tube. In one embodiment, said medical device administers either an internal or external spray to a patient. In another embodiment, said medical device administers either an internal or external gel to a patient.

One embodiment of the present invention contemplates a drug delivery system comprising sirolimus, tacrolimus (FK506) and analogs of sirolimus such as, but not limited to, everolimus (i.e., SDZ-RAD), CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin.

Other derivatives of sirolimus comprising mono-esters and di-esters at positions 31 and 42 have been shown to be useful as antifungal agents (U.S. Pat. No. 4,316,885) and as water soluble prodrugs of rapamycin (U.S. Pat. No. 4,650,803). A 30-demethoxy rapamycin has also been described in the literature (C. Vezina et al. J. Antibiot. (Tokyo), 1975, 28 (10), 721; S. N. Sehgal et al., J. Antibiot. (Tokyo), 1975, 28(10), 727; 1983, 36(4), 351; N. L. Pavia et al., J. Natural Products, 1991, 54(1), 167-177).

Numerous other chemical modifications of rapamycin have been attempted. These include the preparation of mono- and di-ester derivatives of rapamycin (WO 92/05179), 27-oximes of rapamycin (EPO 467606); 42-oxo analog of rapamycin (U.S. Pat. No. 5,023,262); bicyclic rapamycins (U.S. Pat. No. 5,120,725); rapamycin dimers (U.S. Pat. No. 5,120,727); silyl ethers of rapamycin (U.S. Pat. No. 5,120,842); and arylsulfonates and sulfamates (U.S. Pat. No. 5,177,203). Rapamycin was recently synthesized in its naturally occurring enantiomeric form (K. C. Nicolaou et al., J. Am. Chem. Soc., 1993, 115, 4419-4420; S. L. Schreiber, J. Am. Chem. Soc., 1993, 115, 7906-7907; S. J. Danishefsky, J. Am. Chem. Soc., 1993, 115, 9345-9346.

Alternatively, media may also comprise non-sirolimus compounds, such as, but not limited to, antisense c-myc and tumstatin. Other pharmaceutical compounds may be delivered either alone or in combination with sirolimus and analogs of sirolimus, such as, but not limited to, antiinflammatory, corticosteroid, antithrombotic, antibiotic, antifungal, antiviral, analgesic and anesthetic.

Microparticles

One aspect of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Liposomes

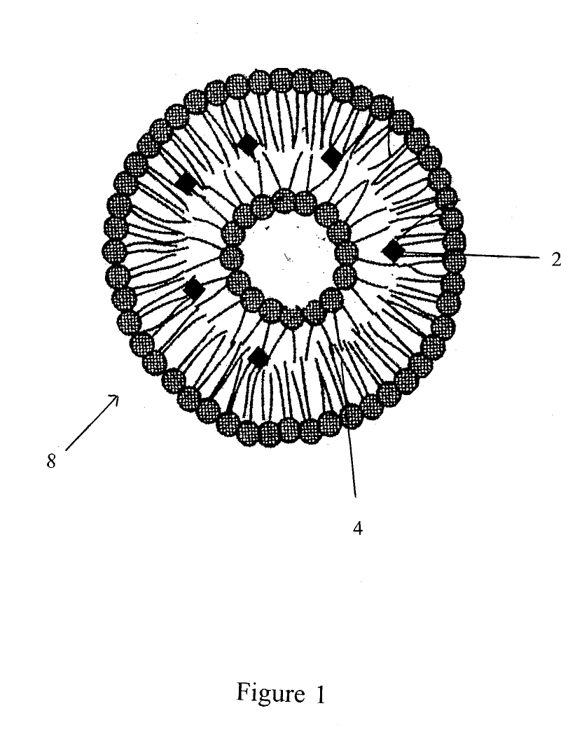

One aspect of the present invention contemplates liposomes capable of attaching and releasing sirolimus and analogs of sirolimus. Liposomes are microscopic spherical lipid bilayers surrounding an aqueous core that are made from amphiphilic molecules such as phospholipids. For example, FIG. 1 demonstrates one liposome embodiment where a sirolimus molecule 2 is trapped between hydrophobic tails 4 of the phospholipid micelle 8. Water soluble drugs can be entrapped in the core and lipid-soluble drugs, such as sirolimus, can be dissolved in the shell-like bilayer. Liposomes have a special characteristic in that they enable water soluble and water insoluble chemicals to be used together in a medium without the use of surfactants or other emulsifiers. As is well known in the art, liposomes form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

The present invention contemplates cationic and anionic liposomes, as well as liposomes having neutral lipids comprising sirolimus and analogs of sirolimus. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of sirolimus and analogs of sirolimus. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel. One of skill in the art should recognize that the terms “microspheres, microcapsules and microparticles” (i.e., measured in terms of micrometers) are synonymous with their respective counterparts “nanospheres, nanocapsules and nanoparticles” (i.e., measured in terms of nanometers). It is also clear that the art uses the terms “micro/nanosphere, micro/nanocapsule and micro/nanoparticle” interchangeably, as will the discussion herein.

Microspheres are obtainable commercially (Prolease®, Alkerme's: Cambridge, Mass.). For example, a freeze dried sirolimus medium is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of sirolimus release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of sirolimus is added to the biodegradable polymer metal salt solution. The weight ratio of sirolimus to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and sirolimus is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and sirolimus mixture are: i) phase separation during a gradual addition of a coacervating agent; ii) an in-water drying method or phase separation method, where an antiflocculant is added to prevent particle agglomeration and iii) by a spray-drying method.

In one aspect the present invention contemplates a medium comprising a microsphere or microcapsule capable of delivering a controlled release of a compound for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Using techniques well known in the state of the art, these microspheres/microcapsules can be engineered to achieve particular release rates. For example, Oliosphere® (Macromed) is a controlled release microsphere system. These particular microsphere's are available in uniform sizes ranging between 5-500 μm and composed of biocompatible and biodegradable polymers. It is well known in the art that specific polymer compositions of a microsphere control the drug release rate such that custom-designed microspheres are possible, including effective management of the burst effect. ProMaxx® (Epic Therapeutics, Inc.) is a protein-matrix drug delivery system. The system is aqueous in nature and is adaptable to standard pharmaceutical drug delivery models. In particular, ProMaxx® are bioerodible protein microspheres that deliver both small and macromolecular drugs, and may be customized regarding both microsphere size and desired drug release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

One embodiment of the present invention contemplates microspheres or microcapsules comprising sirolimus, tacrolimus (FK506) and analogs of sirolimus such as, but not limited to, everolimus (i.e., SDZ-RAD), CCI-779, ABT-578, 7-epi-sirolimus, 7-thiomethyl-sirolimus, 7-epi-trimethoxyphenyl-sirolimus, 7-epi-thiomethyl- sirolimus, 7-demethoxy- sirolimus, 32-demethoxy-sirolimus and 2-desmethyl-sirolimus. Alternatively, microspheres or microcapsules may also comprise non-sirolimus compounds such as, but not limited to, antisense to c-myc and tumstatin. Other, complementary pharmaceutical compounds may be delivered either alone or in combination with sirolimus and analogs of sirolimus, such as, but not limited to, antiinflammatory, corticosteriod, antithrombotic, antibiotic, antifungal, antiviral, analgesic and anesthetic.

In one embodiment, a microparticle contemplated by this invention comprises a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a compound (i.e., for example, sirolimus) is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

Table 1 identifies one embodiment for a microcapsule delivery system for sirolimus. This particular embodiment forms a compound-containing microcapsule bioadhesive gel by contacting the outer microcapsule surface with an adhesive.

TABLE 1
An Exemplary Microcapsule Sirolimus Delivery System

| Component | % by Weight |
|---|---|
| Microcapsule | |
| Plasticized hydrocarbon gel | 80.0 |
| of 60% polyethylene | |
| and 40% mineral oil | |
| Adhesive (mixed with microcapsule) | |
| Guar gum | 6.0 |
| Carboxymethyl cellulose | 6.0 |
| Gum tragacanth | 4.0 |
| Pectin | 3.0 |
| Active Ingredient | |
| Sirolimus | 1.0 |

The bioadhesives of this embodiment allow microcapsules to be placed within the internal mesentery for a sustained period of time for delivery of the compounds contemplated herein. One skilled in the art should realize that various concentrations of sirolimus may be incorporated into the above example (i.e., for example, 0.001%-30%).

In one embodiment, the present invention contemplates microparticles formed by spray-drying a composition comprising fibrinogen or thrombin with sirolimus and analogs of sirolimus. Preferably, these microparticles are soluble and the selected protein (i.e., fibrinogen or thrombin) creates the walls of the microparticles. Consequently, sirolimus and analogs of sirolimus are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area. In one embodiment, the released compound has pharmacologic activity resulting in the reduction of scar tissue formation and/or prevention of tissue adhesion.

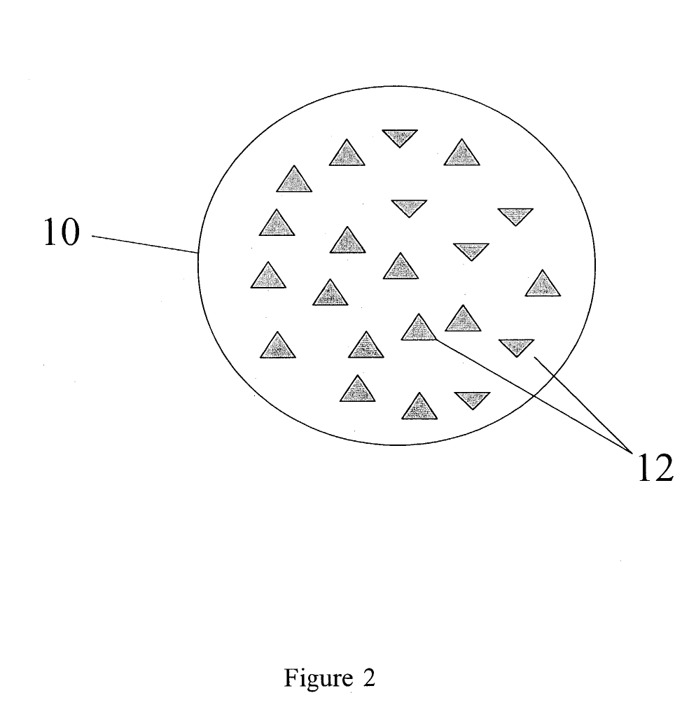

In one embodiment (FIG. 2), the present invention contemplates a microsphere 10 comprising a biocompatible, biodegradable material into which a cytostatic or antiproliferative compound (i.e., sirolimus or an analog of sirolimus) 12 is impregnated (i.e., encapsulated). The compound 12 is contemplated as existing either as fully dissolved or as a colloid.

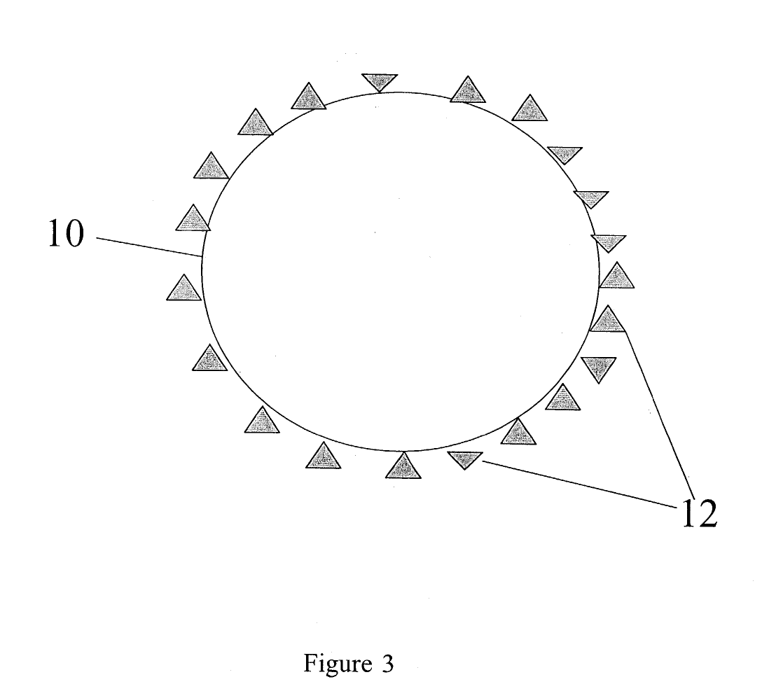

In one embodiment, (FIG. 3), the present invention contemplates a microsphere 10 comprising a biocompatible, biodegradable material into which a cytostatic or antiproliferative compound (i.e., sirolimus or an analog or sirolimus) 12 is adhered to the microsphere 10 surface.

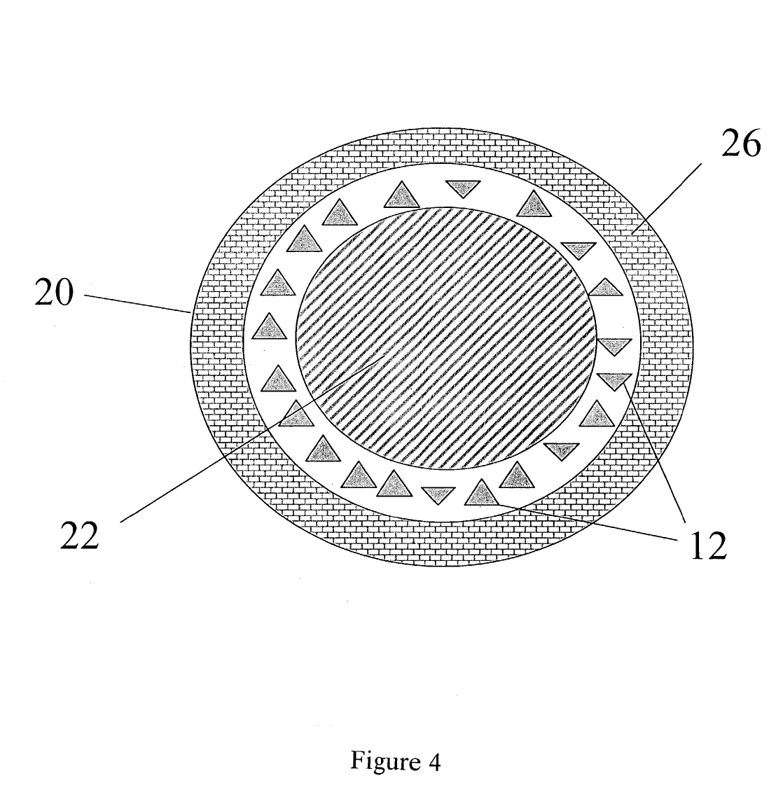

In another embodiment (FIG. 4), the present invention contemplates a microsphere 20 comprising an interior portion 22 comprising a biocompatible, biodegradable material surrounded by a compound layer 12 of a cytostatic, anti-proliferative compound (i.e., sirolimus or an analog of sirolimus) which in turn is surrounded by a second biocompatible, biodegradable material layer 26. Second layer 26 is capable of controlling the rate of release of compound layer 12. Preferably, compound layer 12 is released over a time period of approximately between 1 day and 6 months. In one specific embodiment, compound layer 12 may be contained within a layer 26 or within the interior portion 22.

The diameter of the exemplary microspheres in either FIG. 2 or FIG. 3 should be approximately between 0.1 and 100 microns; preferably 20-75 microns; and more preferably 40-60 microns. One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

The present invention contemplates the combination of microparticles with another medium described herein. For example, a microparticle may be combined with a medium including, but not limited to, a foam, hydrogel, gel or a liquid. In one embodiment a controlled release medium is created. The description of the present invention presents several exemplary embodiments for a variety of mediums. It is not intended for any controlled release medium to be limited to combinations described herein.

Liquid Administration

One aspect of the present invention contemplates the administration of a medium comprising a flowable liquid. Preferably, the liquid media can be administered using a variety of techniques including, but are not limited to, spraying, pouring, squeezing, and the like.

In one embodiment, the present invention contemplates a liquid spray medium comprising liquids, foams, hydrogels, bioadhesives and the like, with or without microparticles. One embodiment of the present invention contemplates spray mediums comprising sirolimus, tacrolimus and analogs of sirolimus. Preferably, a spray may be administered using a catheter directly onto a closed surgical site during an endoscopy procedure such as, but not limited to, laparoscopy or arthroscopy. Alternatively, a spray of said compound may generated by a pressure source (i.e., a spray can or a cylinder comprising a pressure regulator and nozzled tip) to create a droplet spray onto an open surgical site. In another embodiment, a nebulizer (i.e., for example, an atomizer) may also be used to create an aerosol spray. In another embodiment, a spray is administered to an open surgical site.

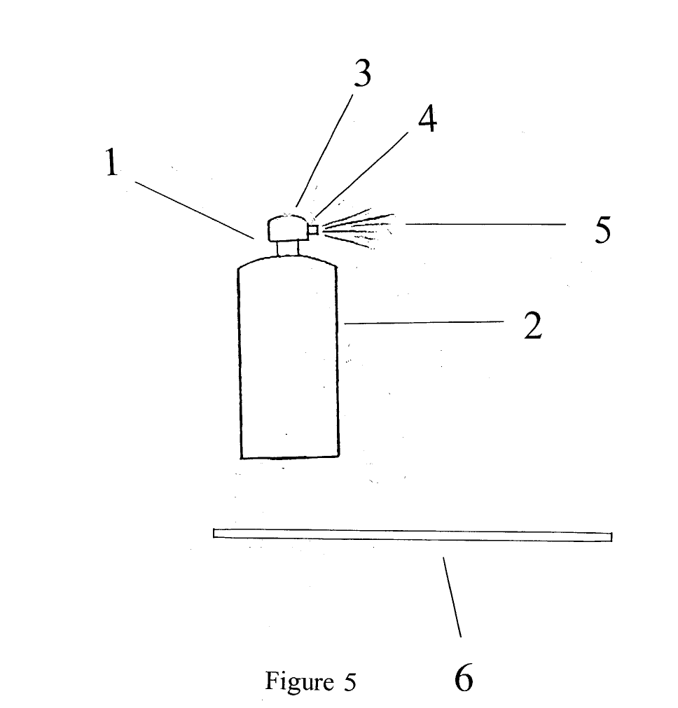

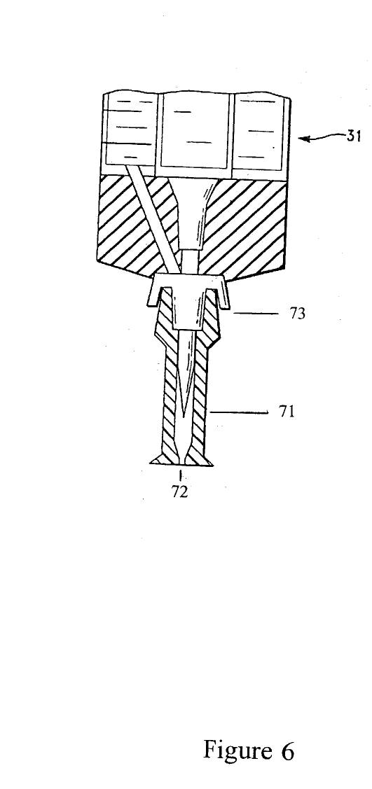

One embodiment of the present invention (FIG. 5) contemplates a pressurized spray can 1 that is capable of spraying a cytostatic anti-proliferative compound (i.e., sirolimus and analogs of sirolimus) into a surgical or wound site. Pressing an actuator button 3 on top of the can body 2 causes the compound spray 5 to exit a nozzle 4. Spray 5 is contemplated to comprise a sirolimus or an analog of sirolimus containing medium selected from the group consisting of an aqueous mixture, microparticles, foam, and bioadhesive. Alternatively, nozzel 4 is attached to a medical tubing 6 or a nebulizer (see FIG. 6) may also be used to spray the compound into the surgical site. One having skill in the art would understand that the present invention is not intended to limit the spraying from a can.

One aspect of the present invention contemplates a method of applying a medium of sirolimus, tacrolimus and analogs of sirolimus, such as, but not limited to liquids, bioadhesives and foams to an internal tissue or organ in an even and controlled manner by a hand-held applicator. In one embodiment, the applicator includes a pump, a tubular extension that is thin enough to pass through an endoscopic lumen, a proximal end of the tubular extension being sealingly connected to the pump, and an applicator tip that attaches to the distal end of the tubular extension. Activation of the pump moves the medium through the tip and onto the internal tissue in an even and controlled manner without contact of the liquid by the pump. In one embodiment, the pump is a micropipetter that includes a hand-held portion having a hand-actuated plunger that does not come in direct, physical contact with the liquid to be dispensed. The device may further include a wound closure device including at least two closure pins extending from the distal end of the tubular extension. In another embodiment, the applicator may be a syringe with a tube extending from the distal end of the syringe. In another embodiment, the tubular extension is large enough for medical personnel to firmly grasp by the hands and apply the medium to an open surgical site. In one embodiment, the applicator comprises two tubular extensions that merge to form a single applicator tip. Preferably, the two tubular extensions contain different media that are applied to the tissue as a single mixture. In one embodiment, the tubular extension contains a powder medium of sirolimus, tacrolimus and analogs of tacrolimus.

In one embodiment, the present invention contemplates a method for spraying a medium comprising sirolimus and analogs of sirolimus onto an open surgical site. Preferably, the sprayed medium comprises a bioadhesive requiring the activation of fibrinogen. Gas-propelled devices are known to spray a first application comprising a first agent capable of gelling or solidifying and then spraying a second application of a second agent that activates said first agent to gel or solidify. Epstein G., Gas Driven Spraying Of Mixed Sealant Agents. U.S. Pat. No. 6,461,361 (herein incorporated by reference). Alternatively, the first and second agents are mixed during spraying such that they are forming a solid matrix as the spray contacts the living tissue. Specifically, one type of sterile-gas ejected bioadhesive spray applicator uses the combination of a protein solution (i.e., thrombin) and a coagulation solution (i.e., fibrinogen). Fukunaga et al., Applicator For Applying A Biocompatible Adhesive. U.S. Pat. No. 5,582,596 (herein incorporated by reference). Alternatively, a metered application of an aerosolized fibrinogen/thrombin bioadhesive is known by using the step-wise mechanical advancement of two syringes in response to a hand-held trigger mechanism shaped similarly to a pistol. Coelho et al., Sprayer For Fibrin Glue. U.S. Pat. No. 5,759,171 (herein incorporated by reference). In another embodiment, microspheres suspended in a liquid carrier are sprayed. In another embodiment, a thermally gelling polymer is sprayed into an open surgical site.

In one embodiment, the present invention contemplates a method for spraying a medium comprising sirolimus and analogs of sirolimus onto a closed surgical site and surrounding tissues. Preferably, application of liquids to a closed surgical site serves as an adjunct to the deployment of a sheet of material by an endoscopic surgical device. In one embodiment, the endoscopic device has multiple openings to dispel a liquid (i.e., saline) during the deployment of the sheet of material. Tilton et al., Instrumentation For Endoscopic Surgical Insertion And Application Of Liquid, Gel And Like Material. U.S. Pat. No. 6,416,506 (herein incorporated by reference). Alternatively, an endoscopic applicator device (i.e., for example, a spray device adapted for use in a laparoscope) is also contemplated to selectively direct a spray application of tissue bioadhesives comprising sirolimus, tacrolimus and analogs of sirolimus. Trumbull, H. R., Laparoscopic Sealant Applicator. U.S. Pat. No. 6,228,051 (herein incorporated by reference). Alternatively, a spray tube or device adapted for use via a catheter in an endoscopic or fluoroscopic device is also contemplated to selectively direct a spray or flow of liquid or gel media comprising cytostatic pharmaceutical compounds (e.g., sirolimus or analogs thereof) to a surgical site.

The present invention contemplates laparoscopic devices capable of delivering a variety of liquid and gel media, including thermoplastic polymers, comprising cytostatic and antiproliferative drugs (i.e., for example, sirolimus, tacrolimus and analogs of sirolimus), biologically-active agents and/or water-insoluble thermoplastic polymers to an area of interest (i.e., for example, an open or closed surgical site). In one embodiment, the invention contemplates using a device ejecting a spray comprising sirolimus and/or analogs of sirolimus under gas pressure that aerosolizes upon exiting a tubular extension rod housing. Fujita et al., Method For Remote Delivery Of An Aerosolized Liquid. U.S. Pat. No. 5,722,950 (herein incorporated by reference). Alternatively, a spray may be generated by slits through the walls of an implanted medical-surgical tube such as a tracheal tube, thoracic or trocar catheter. Preferably, the spray may be an aerosol, coarse spray or liquid stream as determined by the number and size of piercings through the tube wall into the lumen of the tube. Sheridan D., Medico-Surgical Tube Including Improved Means For Administering Liquid Or Gas Treatment. U.S. Pat. No. 5,207,655 (herein incorporated by reference). In one embodiment, the present invention contemplates a spray tip 71, wherein a medium is nebulized by a small orifice 72 (see FIG. 6). Preferably, said spray 71 comprises a luer lock 73 thus allowing compatibility with any standard medical connectors.

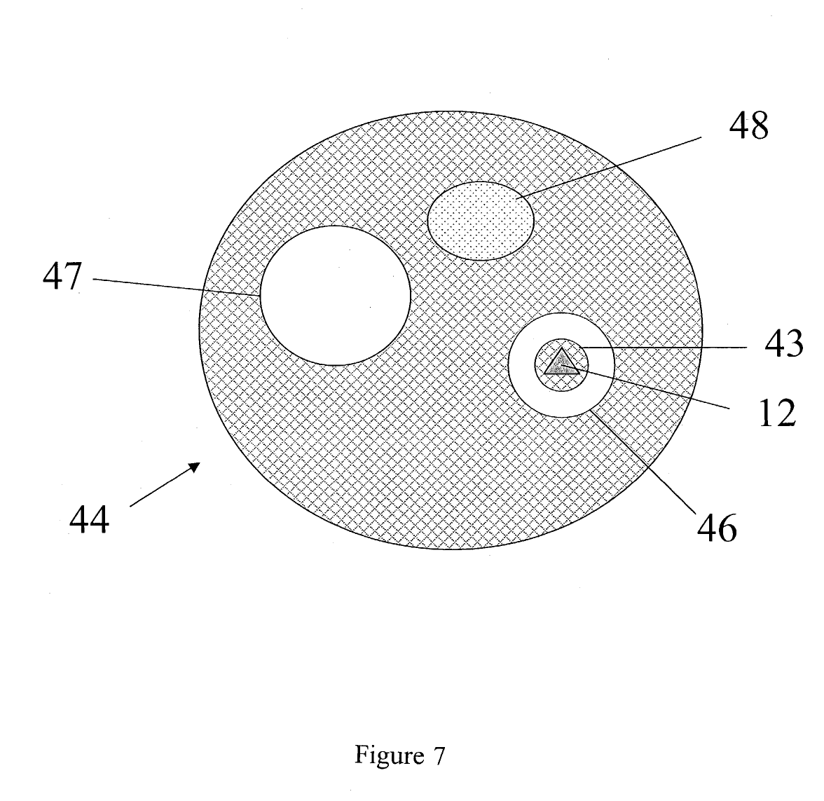

An exemplary endoscope shaft 44 (FIG. 7) may be used during laparoscopic or arthroscopic procedures comprising a viewing optical fiber 48 and a first lumen 47 and a second lumen 46. First lumen 47 could be used for operating a surgical cutting tool (not shown) and second lumen 46 can be used for administering sirolimus and analogs of sirolimus 12 into the surgical site using an endoscopic delivery catheter 43.

In another embodiment, a catheter comprises a common lumen for both a surgical cutting device and for the delivery of a medium comprising sirolimus, tacrolimus and analogs of sirolimus. In one embodiment, sirolimus, tacrolimus or an analog of sirolimus may be administered in the form of a liquid spray, pourable liquids, squeezable liquids, a foam, a gel, a hydrogel, or sheet of material. In another embodiment, the sirolimus compounds are in the form of microparticles as described herein. In one embodiment, a medium comprising said microparticles decreases post-surgical complications by reducing scar tissue formation following either or both a laparoscopy or arthroscopy procedure.

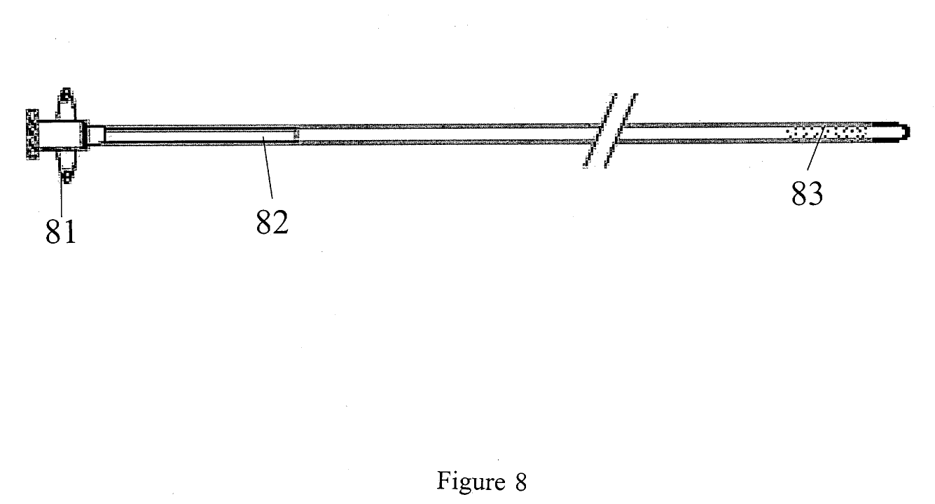

In another embodiment, an endoscopic delivery catheter is inserted through an organ lumen 46 to deliver a medium to a closed surgical site. FIG. 8 shows one embodiment of a typical endoscopic delivery catheter. A female Luer lock adapter 81 is connected to a reservior (not show) that allows a medium comprising sirolimus, tacrolimus or an analog of sirolimus to flow through the catheter lumen 82 and exit the catheter at side ports 83.

The present invention contemplates a method comprising pouring a medium into an open surgical site. In one embodiment, the liquid medium is poured from a hand-held container wherein a flexible tube is capable of directing the flow of the liquid medium. Preferably, said hand-held container includes, but is not limited to, a bottle, a dish, or a mixing tray. In another embodiment, the liquid medium is poured from a fixed container that may be tilted by remote control or manually a medical assistant. Preferably, said fixed container includes, but is not limited to, an applicator tube with a valve for controlling the flow of the medium. In another embodiment, the medium is applied from a tube into an open surgical site. In another embodiment, the medium is applied by squeezing a squeeze bottle.

Bioadhesives

One aspect of the present invention contemplates a bioadhesive medium comprising sirolimus and analogs of sirolimus. Preferably, various embodiments of a bioadhesive medium comprise a biocompatible and biodegradable patch designed for use inside a living organism. In one embodiment, a bioadhesive patch releases a constant compound dose over a period of at least 1 day to 6 months. One of skill in the art would recognize that this embodiment is superior to most conventional transdermal patches currently available for the epidermal layer of the skin. Although it is not necessary to understand the mechanism of an invention it is believed that a bioadhesive patch will heal a wound faster than applying a topical medication that acts locally for only a short time. Additionally, long duration bioadhesive patches do not have the inconvenience and cost of adding more medication for multiple dressing changes. Some bioadhesives are applied using the techniques of liquid administration as defined above.

One embodiment of the present invention contemplates a bioadhesive comprising sirolimus and analogs of sirolimus in combination with a wound healing agent comprising a dental enamel matrix. Gestrelius et al., Matrix Protein Compositions For Wound Healing. U.S. Pat. No. 6,503,539 (herein incorporated by reference). Alternatively, Liquiderm™ adhesive and Dermabond® Topical Skin Adhesive (Closure Medical Corporation) are also compatible with the present invention. Dermabond® adhesive is known as a viable alternative to sutures and staples in closing incisions and lacerations. Liquiderm™ adhesive is brushed on the wound, seals the wound from dirt and germs thereby creating a healing environment.

One embodiment of the present invention contemplates a bioadhesive comprising sirolimus and analogs of sirolimus and an adhesive material consisting of a mixture of hemoglobin and albumin in a solution of glutaraldehyde. Preferably, the coating functions as both a repository for controlled compound release and provides external vascular structural support following surgery. Ollerenshaw et al., Vascular Coating Composition, U.S. Pat. No. 6,372,229 (herein incorporated by reference).

One aspect of the present invention contemplates a method of anastomoses using a bioadhesive comprising sirolimus and analogs of sirolimus. Bioadhesives are known to be useful for anastomoses. Black et al., Sutureless Anastomotic Technique Using A Bioadhesive And Device Therefore, U.S. Pat. No. 6,245,083 (herein incorporated by reference) The impregnation of bioadhesives with sirolimus and analogs of sirolimus, however, to reduce post-surgical scarring is novel.

In one embodiment, the present invention contemplates a method of joining organs, at least one of which has an internal cavity, using a bioadhesive comprising cross-linked proteinaceous materials and a compound selected from the group consisting of sirolimus, tacrolimus and analogs of sirolimus. Preferably, the organs are held in apposition (i.e., by hand or a surgical device) and the organs are joined together using a compound impregnated bioadhesive of the present invention. This joining is facilitated by the creation of apertures by cutting the wall of the organ to allow the introduction of one organ into the other. When the apertures are held together, an anastomosis site is formed at the interface of the two organs to which the bioadhesive of the present invention is applied. For example, a device can be attached to each organ through the use of expandable balloons that become stabilized within the organs when they are inflated. The expandable balloons can be attached to one another by a means extending through the apertures. Hence, an arteriotomy site is dilated while holding the organs to be anastomosed in contact while the bioadhesive is applied. The amount of bioadhesive used is sufficient to seal the joined organs so that the apertures communicate, thereby enabling liquids and compounds to move from one organ to the other through the apertures. Once the bioadhesive sets, the cavities of the two organs can communicate through the joined apertures.

The present invention contemplates a bioadhesive suitable for use in an anastomoses that is non-toxic, has the capability to adhere to biological tissues, reaches stability quickly (typically within about 30 seconds to about 5 minutes), preferably set in wet conditions, bonds to both biological tissues and synthetic materials, and provides sufficient strength to stabilize organs having undergone an anastomosis joining. Preferably, bioadhesive compositions comprising sirolimus and analogs of sirolimus wherein said composition consists of a proteinaceous material and a cross-linking agent are contemplated by this invention for anastomoses. Kowanko N., Adhesive Composition And Method, U.S. Pat. No. 5,385,606 (hereby incorporated herein by reference). The '506 bioadhesive compositions contains two components: i) from 27-53% by weight proteinaceous material; and ii) di- or polyaldehydes in a weight ratio of one part by weight to every 20-60 parts of protein present. To produce the bioadhesive, the two parts are mixed and allowed to react on the surface to be bonded. Bond formation is rapid, generally requiring less than one minute to complete. The resulting adhesion is strong, capable of providing bonds with tear strengths of between 400-1300 g/cm².

Another suitable bioadhesive compatible with the present invention are made by the condensation of a carboxylic diacid with a sulphur-containing amino acid or one of its derivatives. These products contain reactive thiol SH functions which may oxidize to form disulfide bridges, leading to polymers which may or may not be crosslinked. Constancis et al., Adhesive Compositions For Surgical Use. U.S. Pat. No. 5,496,872 (herein incorporated by reference).

One embodiment of the present invention contemplates the extrusion of a double component bioadhesive comprising sirolimus and analogs of sirolimus. In one embodiment, the invention relates to a method of joining, or anastomosing, tubular organs in a side-to-side or end-to-side fashion using bioadhesive. For example, a double component bioadhesive of the '506 patent may be applied through an extruding device having a mixing tip. In one embodiment, a bioadhesive is extruded onto the interface of the two organs in an open surgical field where medical personnel have free and open access to the anastomosis site. In another embodiment, a bioadhesive may be applied by a catheter directed through an endoscope (infra).

The details of the anastomosis embodiment can be exemplified in terms of performing coronary bypass surgery. One embodiment of the present invention contemplates a method for anastomosis of the internal mammary artery (hereinafter “IMA”), also called the internal thoracic artery, to a branch of the left coronary artery comprising; i) isolating an IMA from the chest wall; ii) clamping at a location proximal to the intended site of anastomosis; iii) incising said IMA distal to the intended site of anastomosis; iv) elevating a host artery: v) incising said IMA thus creating a first aperture; vi) isolating said host artery; vii) incising said host artery thus creating a second aperture; viii) inserting a double balloon catheter in said IMA such that said catheter passes through said first aperture and protrudes into said second aperture; ix) inflating a first balloon of said catheter within said host artery such that said second aperture is stabilized; x) positioning said first and second apertures such that they are directly apposed; xi) inflating a second balloon of said catheter within said IMA such that said first aperture is stabilized; xii) applying a bioadhesive comprising sirolimus and analogs of sirolimus around said apposed first and second apertures such that a sufficient strength is reached to maintain an anastomosis; xiii) removing said catheter from said anastomosis; and xiii) ligating said anastomosis.

One embodiment of the present invention contemplates a bioadhesive patch comprising a hydrogel (infra) and a compound selected from the group consisting of sirolimus, tacrolimus and analogs of sirolimus. In a clinical setting, medical personnel would apply the patch containing the compound to a wound, covering it with a bandage. The bandage maintains contact of the hydrogel with the wound and prevents the hydrogel from drying out. Alternatively, a cytostatic and antiproliferative compound (i.e., for example, sirolimus, tacrolimus and analogs of sirolimus) may be incorporated directly into the hydrogel or attached to microparticles, wherein said microparticles are residing within the hydrogel. In one embodiment, a bioadhesive comprising microparticles provide a controlled release medium of said compound.

Bioadhesives are known to comprise fibrin glues, cyanoacrylates, calcium polycarbophil, polyacrylic acid, gelatin, carboxymethyl cellulose, natural gums such as karaya and tragacanth, algin, chitosan, hydroxypropylmethyl cellulose, starches, pectins or mixtures thereof. Alternatively, the adhesives may be combined with a hydrocarbon gel base, composed of polyethylene and mineral oil, with a preselected pH level to maintain gel stability.

In one embodiment an adhesive gel is adjusted to a preselected pH wherein the gel comprises microcapsules. Adhesive biogel system is then placed into a surgical site under conditions such that the active ingredient is delivered.

Foams

One aspect of the present invention contemplates a medium comprising a foam and sirolimus and analogs of sirolimus. It is well known in the art that a foam medium is generally produced from a previously manufactured hydrogel or gel. Therefore, one of skill in the art will understand that any hydrogel medium disclosed herein may be converted into a counterpart foam medium. Many different compositions of foams are known in the art, therefore, the following is only intended as one example of a foam contemplated by the present invention. It is not intended that the present invention be limited by this type of foam.

One embodiment of the present invention contemplates a foam comprising a water-swellable polymer gel and sirolimus and analogs of sirolimus produced by a general process of lyophilizing a gel swollen with water, or by introducing bubbles into the internal of the gel. Preferably, a method for preparing a foam comprising introducing bubbles into the internal of the gel includes processes disclosed in British Patent No. 574,382, Japanese Patent Laid-Open Nos. Hei 5-254029, 8-208868 and 8-337674 and Japanese Unexamined Patent Publication No. Hei 6-510330, and the like. Particularly, when a foam of the water-swellable gel of the present invention is prepared by the process below, there is obtained a foam of a water-swellable polymer gel having higher water absorbability and higher stability as compared to those foams disclosed in those publications.

One example of a method for preparing a foam comprising: i) introducing bubbles into the internal of a gel comprising a compound selected from the group consisting of sirolimus, tacrolimus and analogs of sirolimus, ii) introducing bubbles into an esterified polysaccharide solution or a polyamine solution such that foaming occurs, and iii) contacting said foamed solution with said polyamine solution or said esterified polysaccharide, respectively, to cause gelation. In another example, a method comprises; i) introducing bubbles into a mixed solution of an esterified polysaccharide and a polyamine such that foaming occurs, and ii) completing gelation.

In another embodiment, a method for preparing a foam comprises, i) introducing bubbles into a solution comprising a compound selected from the group consisting of sirolimus, tacrolimus and analogs of sirolimus that is capable of foaming; ii) adding a foaming agent such that a water-insoluble gas is generated and foaming occurs. Preferably, said gas generation results from heating or a chemical reaction using, for instance, but not limited to, ammonium carbonate, azodicarbonamide, p-toluenesulfonyl hydrazide, butane, hexane, and ether. Any method to prepare a foam may further comprise mechanically stirring the solution, thereby diffusing a fed gas into the aqueous solution to foam; and the like.

Any method to prepare a foam may further comprise an ionic or non-ionic surfactant (i.e., a “surface active agent”), which is a bubble-forming agent, as occasion demands, in order to stabilize the foam. In one embodiment, an ionic surfactant includes, for instance, anionic surfactants such as sodium stearate, sodium dodecyl sulfate, α-olefinsulfonate and sulfoalkylamides; cationic surfactants such as alkyldimethylbenzylammonium salts, alkyltrimethylammonium salts and alkylpyridinium salts; and amphoteric surfactants such as imidazoline surfactants. In another embodiment, a non-ionic surfactant includes, for instance, polyethylene oxide alkyl ethers, polyethylene oxide alkylphenyl ethers, glycerol fatty acid esters, sorbitan fatty acid esters, sucrose fatty acid esters, and the like.

Low molecular weight surfactants are known for irritating and denaturing living tissue or a physiologically active substance (i.e., an enzyme or the like). Preferably, non-toxic surfactants are contemplated for foam embodiments of the present invention. Foams contemplated by the present invention comprise a non-toxic surfactant, that are a collection of complex molecules aggregating at the bubble's surfaces. Preferably, such surfactants include, but are not limited to, fats or proteins in edible foams or chemical additives in shaving cream. Although it is not necessary to understand the invention, it is believed that surfactants act by preventing surface tension from collapsing the foam structure by keeping the bubbles separate and repelling water from their surfaces. Foams sprayed from hand-held canisters are capable of expanding to about 100 times their liquid volume as air is drawn into the spray. An advantage of a foam over a liquid is that the foam fills crevices and other elusive hiding places as the expansion process occurs.

Although it is not necessary to understand an invention, it is believed that an esterified polysaccharide itself exhibits amphipathic properties and functions as a bubble-forming agent for stabilizing the gas-liquid interface. Consequently, in some embodiments a surfactant may not be necessary in the presence of esterified polysaccharides. Since an esterified polysaccharide has reactivity in addition to the amphipathic property, the esterified polysaccharide can be referred to as a “reactive surfactant polysaccharide.”

In some embodiments, surfactants may also be, but not limited to, a protein such as albumin, gelatin or albumin, or lecithin.

In one embodiment, a method for preparing a foam further comprises adding a bubble stabilizer. Preferably, bubble stabilizers include, but are not limited to, a higher alcohol such as dodecyl alcohol, tetradecanol or hexadecanol; an amino alcohol such as ethanolamine; a water-soluble polymer such as carboxymethyl cellulose; and the like. Alternatively, bubble stabilizers may be polysaccharides comprising natural polysaccharides such as agarose, agaropectin, amylose, amylopectin, arabinan, isolichenan, curdlan, agar, carrageenan, gellan gum, nigeran and laminaran. While it is not necessary to understand an invention, it is believed that bubble stabilizers prevent the disappearance of bubbles prior to the completion of crosslinking.

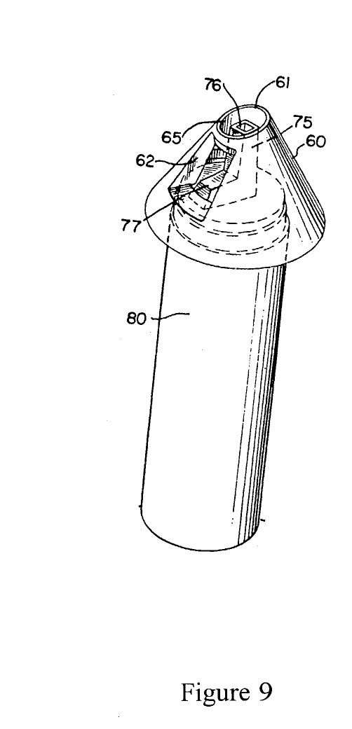

In one embodiment, the present invention contemplates a pressurized canister comprising a foam and a compound, such as, but not limited to, sirolimus, tacrolimus and analogs of sirolimus. As depicted in FIG. 9 a pressurized foam canister 80 has a generally cylindrical body. Foam canister 80 includes a movable dispensing valve 75 coupled thereto that is accessed by finger aperature 62. Valve 75 is constructed in accordance with conventional fabrication techniques and defines an upwardly extending valve passage 76 and a laterally extending ledge 77. Valve 75 is operable to discharge the pressurized foam contents through valve passage 76. A generally conical cap 60 defines a nozzel aperture 61 at its apex and a downwardly extending nozzle passage 65. Valve 75 also extends partially into nozzle passage 65 within cap 60.

Gels

A hydrogel medium comprises a three-dimensional networks of hydrophilic polymers, either covalently or ironically cross-linked, which interact with aqueous solutions by swelling and reaching an equilibrium. Compounds, such as, but not limited to, sirolimus and analogs of sirolimus, can be added to a hydrogel medium during the manufacturing process. Hydrogel medium technology encompasses many different types of compositions, therefore, the term “hydrogel” does not refer to any specific composition but identifies a composition having specific properties. For example, hydrogels may provide controlled release of drug compounds included in them by providing physical barriers or through chemical attachment of the drug to the hydrogel.

Hydrogels are primarily characterized by having an ability to swell in aqueous solutions. Swelling ratios and solubility are controlled by the specific composition of the hydrogel. Higher swelling ratios result in a greater release rate of an incorporated compound that is attached to or contained within the hydrogel. Although it is not necessary to understand the mechanism of an invention, it is believed that a high swelling ratio results in, more open structure within the hydrogel and more closely mimics living tissue, therefore facilitating the process of diffusion between the hydrogel and the tissue. High swelling ratios are also related to the overall hydrophilicity of the hydrogel composition, and provide for better absorptive properties.

In one embodiment, the present invention contemplates a hydrogel matrix having the capability to provide controlled release of a compound prepared by: i) adding heparin (400 mg, 0.036 mmole) to 750 mls of double distilled water at 4° C.; ii) adding human serum albumin (550 mg, 0.0085 mmole) 1.0 ml double distilled water at 4° C., and iii) adding N-(3-dimethylaminopropyl)- N-ethylcarbodiimide (i.e., EDC solution; 94 mg) to 250 ml double distilled water at 4° C. The heparin solution, along with the 1 ml of the albumin solution are first mixed within a 2 ml polyethylene-polypropylene syringe containing a small stir bar and a desired concentration of a compound (i.e., for example, sirolimus). Subsequently, the EDC solution is added to form the final mixture. All steps are carried out at 4° C.

After 24 hours, a hydrogel is removed from the syringe by swelling as the syringe is placed in toluene. After the albumin-heparin hydrogel is extruded from the syringe, the hydrogel is then equilibrated with phosphate buffered saline to remove uncoupled material.

The release rate of the attached compound may be controlled by varying the amount of heparin present in the matrix.

One embodiment of the present invention contemplates a hydrogel laminate comprising sirolimus and analogs of sirolimus and crosslinked hydrophilic-adhesive polymers. Such compositions form absorbent products such as bandages. Preferably, hydrogel polymers are generally synthetic polyvinylpyrrolidone, polyethyleneoxide, acrylate, and methacrylate and copolymers thereof. Kundel, Hydrogel Laminate, Bandages and Composites And Methods For Forming The Same, U.S Pat. No. 6,468,383 (herein incorporated by reference). Alternatively, hydrogels compatible with the present invention may be formed by crosslinking carbohydrates, such as dextran, with maleic acid or hyaluronic acid with polyvinyl chloride. Kim et al., Dextran-Maleic Acid Monoesters And Hydrogels Based Thereon. U.S. Pat. No. 6,476,204; Giusti et al., Biomaterial Comprising Hyaluronic Acid and Derivatives Thereof In Interpenetrating Polymer Networks (IPN). U.S. Pat. No. 5,644,049 (both herein incorporated by reference).

Another embodiment contemplates a hydrogel medium comprising hyaluronic acid capable of controlled release of sirolimus and analogs of sirolimus. While these compositions are disclosed as topical and injectable polymer solutions, the present invention contemplates a hyaluronic acid polymer solution within a hydrogel to time-release the delivery of compounds, such as, but not limited to, sirolimus and analogs of sirolimus within the body. Drizen et al., Sustained Release System. U.S. Pat. No. 6,063,405 (herein incorporated by reference).

One aspect contemplated by the present invention comprises a hydrogel medium comprising sirolimus or analogs of sirolimus, wherein said hydrogel has a controlled gelation time. In one embodiment, the hydrogel is made of one or more synthetic and/or natural water-soluble polymers, and one or more divalent or multivalent cation containing or releasing compounds. At least one of the polymer monomers is an acid or a salt thereof that is capable of reacting with the divalent or multivalent cation to form intermolecular polymer ionic crosslinks. Such hydrogels are discussed in detail relating to use for tissue culture scaffolding. Ma P. X., Ironically Crosslinked Hydrogels With Adjustable Gelation Time. U.S. Pat. No. 6,497,902 (herein incorporated by reference). Specifically, controlled gelation time is taught as a function of: i) cation solubility; ii) cation concentration; iii) mixture/ratio of cation containing compounds; iv) polymer concentration; and v) gelation temperature.

In one embodiment, the present invention contemplates the administration of a hydrogel comprising sirolimus and analogs of sirolimus to an open surgical site. In another embodiment, the present invention contemplates the administration of a hydrogel comprising sirolimus and analogs of sirolimus to a closed surgical site via a catheter (i.e., during laparoscopic procedures) that transitions into a gel upon contact with living tissue. In one embodiment, a micelled hydrogel core serves as a reservoir of sirolimus and analogs of sirolimus. In another embodiment, a hydrogel comprises microparticles attached to sirolimus and analogs of sirolimus. Sirolimus and analogs of sirolimus are contemplated by the present invention as pharmacologically effective in reducing scar tissue and improving the healing of wounds or surgical incisions.

One embodiment of the present invention contemplates a controlled release hydrogel medium comprising sirolimus and analogs of sirolimus formed by crosslinking a protein (i.e., albumin, casein, fibrinogen, γ-globulin, hemoglobin, ferritin and elastin) with a polysaccharide (i.e., heparin, heparin, chondroitin sulfate and dextran). Determinative factors regulating compound release from a hydrogel medium is: i) gel composition; ii) crosslinking degree; and iii) gel surface treatments. Specifically, it is known that hydrogel releasable compounds include hormones, cytostatic agents, antibiotics, peptides, proteins, enzymes and anticoagulants. Feijen J., Biodegradable Hydrogel Matrices For the Controlled Release Of Pharmacologically Active Agents. U.S. Pat. No. 4,925,677 (herein incorporated by reference). Alternatively, controlled release of compounds from a hydrogel medium contemplated by the present invention is possible by inserting hydrolyzable spacers between polymer crosslinks. In one embodiment, a hydrogel degradation rate is contemplated to be modified to provide dissolution rates from 1 day to 6 months. Hennink et al., Hydrolyzable Hydrogels For Controlled Release. U.S. Pat. No. 6,497,903 (herein incorporated by reference).

Alternatively, a hydrogel medium may act as a compound repository in their own right wherein diffusion creates a time-release delivery of a compound into the surrounding tissue. Kennedy et al., Semisolid Therapeutic Delivery System And Combination Semisolid, Multiparticulate, Therapeutic Delivery System. U.S. Pat. No. 6,488,952 (herein incorporated by reference). In one embodiment, the present invention contemplates a hydrogel comprising a liposome comprising sirolimus and analogs of sirolimus covalently attached to a medical device, such as, for example, a wound dressing. Preferably, a hydrogel medium contemplated by this invention comprises a material selected from the group consisting of gelatins, pectins, collagens and hemoglobins. DiCosmo et al., Compound Delivery Via Therapeutic Hydrogels, U.S. Pat. No. 6,475,516 (herein incorporated by reference). In one particular embodiment, a hydrogel comprises microparticles containing sirolimus or an analog of sirolimus.

One embodiment of the present invention contemplates a method providing a medical device comprising a catheter capable of placing a hydrogel comprising sirolimus and analogs of sirolimus at a closed surgical site. Sahatjian et al, Compound Delivery, U.S. Pat. No. 5,674,192 (herein incorporated by reference). Preferably, said hydrogel comprises a second compound designed as a wound healing agent such as, but not limited to, dental enamel matrix. Gestrelius et al., Matrix Protein Compositions For Wound Healing. U.S. Pat. No. 6,503,539 (herein incorporated by reference).

One aspect of the present invention contemplates thermo-reversible gel technology based on the use of biocompatible poloxamers made up of polyoxyethylene and polyoxypropylene units. Preferably, these poloxamers comprise any polymer or copolymer sold under the trademarks Pluronics® or Tetronics®. A Tetronic® gel-forming macromer contains four covalently linked polymeric blocks, wherein at least one polymeric block is hydrophilic, linked by a common crosslinkable group and is disclosed as a thermal gelling drug delivery device. U.S. Pat. No. 6,410,645 To Pathak et al. (herein incorporated by reference). These gels are discussed as having thermosensitivity and lipophilicity, and may be used to administer drugs and tissue coatings for medical applications. Other Tetronic® polyols, having hydrophobic polymeric blocks, are known as drug delivery devices. U.S. Pat. Nos. 4,474,751; 4,474,752; 4,474,753; and 4,478,822 To Haslam et al. (all herein incorporated by reference).

In one embodiment, poloxamer 407 (i.e., Pluronics® F-127) is a primary ingredient and can be manufactured in a variety of formulations with specific physical and chemical properties. The most significant physical characteristic of thermo-reversible gels is an ability to change from a liquid to a gel upon warming to body temperature. This characteristic allows for manipulation of the polymer product in its liquid state and conversion to a desired solid state (i.e., a gel) in or on the body of the patient. One specific advantage of administering a thermal gel in a liquid state includes molding to body/tissue contours before gelling in place. Thus, the thermal gel maintains contact with the tissue surface and serves as a physical, protective barrier in addition to serving as a carrier for drug delivery to adjacent tissues. Typically, thermal gels are comprised of materials known to be non-toxic, non-irritating and pharmacologically inert. Furthermore, thermal gels dissolve in the body and are cleared by the normal excretory processes.

The present invention contemplates a biocompatible thermal gel medium comprising sirolimus and analogs of sirolimus attached to microparticles. In one embodiment, the microparticles are capable of controlled release of the sirolimus and analogs of sirolimus. In one embodiment, the thermal gel medium comprises a polymer gel, such as, but not limited to Flogel® (Alliance Pharmaceutical Corp). Preferably, polymer gels such as FloGel® are applied to tissues and organs as a chilled liquid that solidifies into a gel as it warms to body temperature, creating a physical barrier that holds the microspheres in place while the thermal gel and the microspheres bioerode and the cytostatic compound is released such that excess scar tissue is prevented.

Xerogels

One aspect of the present invention contemplates a device and method for long-term controlled release of a medium comprising sirolimus, tacrolimus and analogs of sirolimus. In one embodiment, the medium comprises a xerogel, exemplified by the commercially available product Xerocell™ (Gentis, Berwyn, Pa.). Xerogels comprise a plurality of microscopic air bubbles suffused in a glassy matrix. In one embodiment, the present invention contemplates a controlled release medium comprising a xerogel and sirolimus, tacrolimus and/or analogs of sirolimus. Preferably, the xerogel allows complete control over a controlled release profile from approximately a few hours to more than a year.

One aspect of the present invention contemplates a method comprising placing a xerogel comprising sirolimus, tacrolimus and analogs of sirolimus at or near a surgical site. In one embodiment, said surgical site heals over and around the xerogel. In one embodiment, the xerogel provides a controlled release the sirolimus, tacrolimus and/or analogs of sirolimus such that surgical scar and/or adhesion tissue formation is reduced.

Surgical Dressings/Tapes

One aspect of the present invention contemplates surgical dressings and surgical tapes comprising a medium and sirolimus and analogs of sirolimus. Illustrative examples of such dressings and tapes include, but are not limited to, sheets of material, surgical swabs, gauze pads, closure strips, compress bandages, surgical tape, etc. For example, one embodiment of the present invention contemplates a laminated composite comprising a first nonwoven fiber layer, an elastic layer, a melt blown adhesive fiber layer, and a second nonwoven fiber layer, wherein said composite comprises sirolimus and analogs of sirolimus impregnated into said second nonwoven layer. Menzies et al., Laminated Composites, U.S. Pat. No. 6,503,855 (herein incorporated by reference).

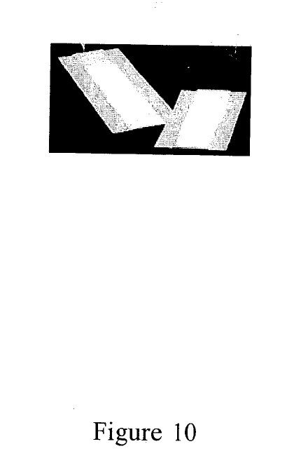

In one embodiment (FIG. 10), the present invention contemplates a biocompatible sheet of material or mesh comprising sirolimus and analogs of sirolimus impregnated (i.e., attached) into, coated onto or placed onto a material sheet or mesh. Such sheets of material may placed between internal body tissues to prevent the formation of post-operative adhesions and/or scar tissue. In one embodiment, the sheet of material is biodegradable (Surgicel™, Johnson & Johnson). In another embodiment, said sheet of material comprises a surgical suture. In another embodiment said sheet of material comprises a surgical staple. In another. embodiment, said sheet of material comprises an eye buckle. In another embodiment, said sheet of material comprises a cylindrical tube.

In one embodiment, the present invention contemplates a moist dressing product comprising a medium of sirolimus and analogs of sirolimus. Preferably, these dressings consist of a flexible film having a polyurethane gel core. Although it is not necessary to understand the mechanism of an invention, it is believed that moist dressing products reduce the formation of a hard scab and reduces the likelihood of scarring. For example, these dressings may include, but are not limited to, those currently marketed as Elastoplast® (Active Gel Strips; Beiersdor, Inc.).

In one embodiment, the present invention contemplates a semipermeable membrane formed from a unique blend of silicone and polytetrafluoroethylene (PTFE) and a medium of sirolimus and analogs of sirolimus. Although it is not necessary to understand the mechanism of an invention, it is believed that the PTFE provides an internal reinforcing mechanism, thereby creating very thin sheets of soft silicone with significantly enhanced physical strength. For example, these dressings may include, but are not limited to, those currently marketed as Silon-IPN™ (Bio Med Sciences).

In one embodiment, the present invention contemplates dressings comprising a polyurethane membrane-matrix on a semi-permeable thin-film backing and sirolimus and analogs of sirolimus. Preferably, the hydrophilic membrane contains a cleanser, a moisturizer and a super-absorbent starch co-polymer. Although it is not necessary to understand the mechanism of an invention, it is believed that eliminates the need for manual debridement and cleaning during dressing changes is eliminated and reduces patient discomfort and the time and cost of dressing changes. For example, these dressings may include, but are not limited to, those currently marketed as PolyMem® (Ferris Mfg., Inc.).

In one embodiment, the present invention contemplates closure strips comprising sirolimus and analogs of sirolimus. Preferably, said skin closures are useful in a method to provide skin closure following intra-abdominal operations. Alternatively, these closures may be used with any traditional sutures or sutures coated with sirolimus and analogs of sirolimus. Although it is not necessary to understand the mechanism of an invention, it is believed that the advantages of skin closures contemplated by the present invention are: i) lower rates of infection and over-all morbidity; ii) a lower cost; iii) a reduction in time in the operating room when compared with conventional methods; and iv) avoidance of foreign body granulomas, strangulation, tissue necrosis and cellulitis. Pepicello et al., Five Year Experience With Tape Closure Of Abdominal Wounds. Surg Gynecol Obstet 169:310-4 (1989).

Marker Agents

The present invention contemplates incorporating any color as a marker agent into any medium discussed herein. In one embodiment, a desired colored medium comprises a marker comprising a colored dye or stain such as the blue dye “Brilliant Blue R”, also known as “Coomassie™ Brilliant Blue R-250” (distributed as “Serva Blue”; Serva) The resulting medium has a blue color that provides a good contrast to the color of body tissues, making the medium easy to see during surgery. In another embodiment, the present invention contemplates a gel, film or spray made up of two liquids which comprise sirolimus and analogs of sirolimus, that when sprayed together, solidify to form a bright colored material which breaks down gradually over about a week.

One embodiment of the present invention contemplates a method providing a biocompatible and biodegradable microsphere or hydrogel having a coloring marker agent such that medical personnel are capable of adequately covering an intended region where scar tissue and/or adhesions might form.

The present invention also contemplates incorporating a radio-opaque marker into any medium discussed herein. In one embodiment, said radio-opaque marker comprises a barium compound. In one embodiment, said radio-opaque marker is visualized using X-ray fluoroscopy.

For any of the applications described herein, the systemic application of one or more of the cytostatic anti-proliferative agents that have been described could be used conjunctively to further minimize the creation of scar tissue. The systemic application could be by mouth, by injection, or by any other well known means for placing a compound systemically into a human body.

Although only the use of certain compounds, such as, sirolimus and analogs of sirolimus, and those capable of binding to the mTOR protein and/or interrupting the cell cycle in the G0 or G1 phase has been discussed herein, it should be understood that supplemental pharmaceutical compounds may be provided to improve the outcome for the patients. Specifically, an antibiotic, and/or analgesic, and/or anti-inflammatory agent could be added to prevent infection and/or to decrease pain. It is further understood that any patient in whom sirolimus and analogs of sirolimus is used in combination with at least one supplemental pharmaceutical compound may have an improved response if sirolimus and analogs of sirolimus is also given as a conventional administration.

Various other modifications, adaptations, and alternative designs are of course possible in light of the above teachings. Therefore, it should be understood at this time that within the scope of the appended claims, the invention can be practiced otherwise than as specifically described herein.

Experimental

The following examples serve to illustrate certain preferred embodiments and aspects of the present invention and are not to be construed as limiting the scope thereof.

In the experimental disclosure which follows, the following abbreviations apply: g (gram); mg (milligrams); μg (microgram); M (molar); mM (milliMolar); μM (microMolar); nm (nanometers); L (liter); ml (milliliter); μl (microliters); ° C. (degrees Centigrade); m (meter); sec. (second).

### EXAMPLE I

### A Controlled Release Microsphere for Hydrophobic Compounds

This example describes the production of a microsphere capable of administering sirolimus in controlled release manner.

A controlled release microsphere pharmaceutical composition is made that is burst-free and provides a sustained programmable release of a sirolimus compound over a duration of 24 hours to 100 days made in accordance with U.S. Pat. No. 6,447,796 To Vook et al. (herein incorporated by reference). These microspheres are particularly suited for hydrophobic drugs by using a blend of end-capped and uncapped biocompatible, biodegradable poly(lactide-co-glycolide) copolymers (PLGA). The end-capped polymers have terminal residues functionalized as esters and the uncapped polymers have terminal residues existing as carboxylic acids.

PLGA copolymers contemplated by this Example has a molecular weight ranging from 10 to 100 kDa are in a 50:50 ratio, although one skilled in the art would understand that other ratio's are also possible. Briefly, well known solvent evaporation techniques are used to prepare sirolimus/PLGA microspheres in a range of 0.1-2.0 mg of sirolimus per 100 mg PLGA. The evaporation technique is expected to result in microsphere core loads of 10%, 20%, 40%, and 50% of a theoretical maximum. Empirical testing is performed to determine the proper ratios of sirolimus and PLGA copolymer concentrations that result in these predicted core loading efficiencies.

For example, a useful protocol is as follows:

1) Pre-heat water bath to 15° C.

2) Prepare 1% poly-vinyl alcohol solution in distilled water.

3) Prepare a 1% poly-vinyl alcohol solution in methylene chloride-saturated distilled water.

3) Co-dissolve appropriate amounts of sirolimus and PLGA in 3.5 g methylene chloride.

4) Add the PLGA-sirolimus solution to 25 ml of the 1% poly-vinyl alcohol solution in methylene choloride-saturated distilled water.

5) Homogenize the mixture at 10,000 rpm for 30 seconds in a 50 ml centrifuge tube.

6) Add the homogenized mixture to 500 ml of the 1% poly-vinyl alcohol solution in distilled water.

7) Stir at 650 rpm for ½ hour at 15° C.

8) Stir at 650 rpm for 4 hours at 25° C.

9) Collect microspheres by filtration.

10) Wash collected microspheres.

10) Vacuum dry collected microspheres overnight.

Microspheres are expected to show an average diameter range of between 2.5-200 μm, prefereably between 4.0-75 μm, and more preferably between 5.0-10.0 μm. Release rates in relationship to core loading capacity are expected as: i) 40.19% sirolimus release in 10 days using a 10% core load; ii) 71.58% sirolimus release in 6 days using a 20% core load; iii) 48.09% sirolimus release in 6 days using a 40% core load; and iv) 39.84% sirolimus release in 6 days using a 50% core load. Administration of sirolimus containing microspheres prepared according to this method may be performed by any method contemplated herein.

### EXAMPLE II

### Liposome Encapsulation

This example describes a method to prepare liposomes that encapsulate sirolimus.

Multilammelar vesicles (i.e., liposomes) are prepared from egg phosphatidylcholine (EPC) and cholesterol (Ch) (ratio 4:3). Specifically, a preliposomal lipid film will be obtained by drying under nitrogen atmosphere a mixture of EPC (14.4 mg=18.3 μmoles), 5.6 mg cholesterol (13.7 μmoles) and 0.1 mg sirolimus in a nonpolar organic solvent such as dichloromethane or chloroform. The resulting dry lipid film is then converted into a liposomal suspension of the multilammelar vesicles encapsulating the sirolimus by hydrating the dry lipid film with 1 ml isotonic phosphate buffer pH 8.1, and smooth shaking of the suspension during formulation. Finally, sorbitol is then added to the suspension in an amount of 1% wt/volume, at a molar ratio of sorbitol to phospholipid of 3:1.

The final liposomal suspension is then freeze dried at −25° C. by direct immersion in denatured ethanol. The association (i.e., encapsulation efficiency) of sirolimus with 1 ml of the liposomal suspension with 1 ml of the liposomal suspension before and after freeze drying is expected to be approximately 80%.

### EXAMPLE III

### A Hydrogel Composition

This example provides a composition where sirolimus is incorporated into a hydrogel such that the sirolimus is released by diffusion.

This hydrogel composition will incorporate and retain significant amounts of H₂O, and eventually reach an equilibrium content in the presence of an aqueous environment. Glyceryl monooleate (i.e., GMO) is described herein, however on skilled in the art will recognize that many polymers, hydrocarbon compositions and fatty acid derivatives having similar physical/chemical properties with respect to viscosity/rigidity are capable of producing hydrogels for purposes of this invention.

First, the GMO is heated above its melting point (i.e., 40° C.-50° C.). Second, a warm aqueous-based buffer (i.e., an electrolyte solution) such as phosphate buffer or normal saline or a semi-polar solvent containing the desired concentration of any sirolimus suspension or water soluble sirolimus derivative as discussed herein, is added to produce a three-dimensional hydrogel composition.

The selection of GMO as a gel polymer is advantageous due to its amphipathic properties. Specifically, GMO will provide a predominantly lipid-based hydrogel, thereby incorporating lipophilic compounds such as sirolimus.

At room temperature (i.e., 20° C.-25° C.) this hydrogel will exist in a lamellar phase consisting of approximately 5%-15% H₂O and 95%-85% GMO. This lamellar phase is a moderately viscous fluid, which is easily manipulated, poured and injected. However, when this hydrogel is exposed to physiologic temperature and pH (i.e., approximately 37° C. and pH 7.4) a cubic phase (i.e., a liquid crystalline gel) results consisting of approximately 15%-40% H₂O and 85%-60% GMO and is expected to have an equilibrium water content (i.e., maximum water content in the presence of excess water) of approximately 35%-40% by weight. This cubic phase is highly viscous and will exceed 1.2 million centipoise (cp).

### EXAMPLE IV

### A Thermoreversible Gel

This example demonstrates that sirolimus may be incorporated into a thermoreversible gel polymer composition having internal micellular components sufficient for controlled release.

This polymer composition is represented by the composition trademarked as Flogel® (Alliance Pharmaceuticals; San Diego, Calif.) and comprises a polyoxyethylene-polyoxypropylene block copolymer having the formula HO(C₂ H₄ O)_{b} (C₃ H₆ O)ₐ (C₂ H₄ O)_{b} H, wherein a is an integer such that the hydrophobe base represented by (C₃ H₆ O)ₐ has a molecular weight of at least about 900, preferably at least about 2500, most preferably at least about 4000 average molecular weight, as determined by hydroxyl number. Similar polymer compositions may also be produced having a polyoxyproplyene hydrophobe base average molecular weight of about 4000, a total average molecular weight of about 12,000 and containing oxyethylene groups in the amount of about 70% by weight of the total weight of the copolymer. A preferred copolymer is a tri-block copolymer containing two polyoxyethylene blocks flanking a central polyoxypropylene block and is sold under the trademark Pluronic® F-127 (BASF Corp, Parsippany, N.J.).

In this example, Pluronic® F-127 mixed with sirolimus, as discussed herein, is placed in water, and the Pluronic® F-127 self-assembles so as to remove contact between the polyoxypropylene groups and water (i.e. self-assembly is driven by a hydrophobic effect). These self-assembled units are termed micelles within which are trapped sirolimus drug molecules. The structure of the micelles and the interactions between them is strongly dependent on temperature. A large increase in solution viscosity (i.e. gel-phase formation) is noted with increasing temperature due to the organization of the micelles into a three-dimensional cubic array (see Example III). This gelation time may be controlled by the addition of a modifying polymer including, but not limited to, cellulose derivatives.

The Pluronic® F-127-sirolimus solution is maintained at +4° C. until the time of use. When the chilled solution is placed on or within a living tissue the solution will gel to form a solid matrix on the surface of the tissue. During the subsequent controlled dissolution of the matrix, the sirolimus will be slowly released into the immediate environment to prevent scar tissue and adhesion formation. The dissolution rates of thermoreversible gels may be controlled by compounds including, but not limited to, fatty acid soap derivatives. It is expected that the gelled matrix begins dissolution during the first day after administration and is completely dissolved following twenty-one days after administration.

### EXAMPLE V

### A Fibrin-Based Microparticle Bioadhesive

This example described the preparation of a powdered fibrin bioadhesive containing a sirolimus compound. Specifically, the composition comprises microparticles containing a fibrinogen-thrombin matrix and sirolimus. This protocol entails the preparation of two separate powders (i.e, a fibrinogen powder and a thrombin powder) that are mixed together just prior to use. U.S. Pat. No. 6,113,948 To Heath et al. (herein incorporated by reference).

Briefly, the first powder comprises fibrinogen and sucrose and the second powder comprises thrombin, CaCl₂, sirolimus and mannitol. Fibrinogen is first formulated with 600 mg sucrose. The resulting composition is then spray-dried using a Mini Spray Dryer with a collecting vessel under the following conditions:

| | |
|---|---|
| Inlet Temperature: | 100° C. |
| Outlet Temperature: | 65° C. |
| Atomisation Pressure: | 1.0 bar |
| Atomisation Type: | Schlick 970/0 |
| Feed Rate: | 1 g/min |

A 20% final excipient loading is expected along with a fibrinogen theoretical activity of 10 mg/100 mg. This indicates a full retention of the fibrinogen bioactivity.

The second powder is prepared by dissolving 1 g D-mannitol in 10 ml of 40 mM CaCl₂ with any soluble form of sirolimus, as described herein, at a concentration sufficient to obtain a final concentration of 2 mg/15 cm² of tissue surface. The resultant solution is used to reconstitute 1 vial of thrombin. The spray-drying conditions are essentially the same as for the first powder, except that the outlet temperature is 62° C., and the feed rate is reduced to 0.75 g/min.

A thrombin clotting assay should reveal a thrombin activity of 91.86 units/100 mg that will compared favorably with the theoretical activity, of 93 units/100 mg. This indicates full retention of thrombin bioactivity.

The first and second microparticle powders are then mixed to form a 50:50 blend in a glass vial by placement on a roller mixer for 20 minutes. This activated mixture is then applied to a biological tissue.

### EXAMPLE VI

### A Dual Component Bioadhesive with PLGA Microsspheres

This example describes a composition for a sirolimus-eluting bioadhesive consisting of proteinaceous materials and a cross-linking agent.

Dry plasma solids are obtained by lyophilizing fresh frozen human plasma. Thereafter, water is added to this solid to produce a viscous solution containing 45% of solids by weight to create Solution A. Sirolimus-eluting microspheres, prepared in accordance with Example I, are then added to Solution A. Solution B is prepared by creating an aqueous 10% (w/w) glutaraldehyde mixture. The bioadhesive properties may be tested by lightly spraying two rectangular (i.e., 2.5 cm×2.5 cm) blocks of meat with Solution B on the surfaces to be bonded. The surfaces are then coated with Solution A to a thickness of 1-2 mm, and again sprayed with Solution B. This process will result in a ratio of Solution A to Solution B of 7 to 1 by weight. The surfaces are then joined within about 10 seconds of the application of Solution A and held in position until cure was complete, generally 15-60 seconds, depending on temperature and on the effectiveness of mixing Solution A and Solution B.

If the sequence of application of Solution A and Solution B is reversed or if Solution A and Solution B are applied simultaneously or if Solution A and Solution B are pre-mixed immediately prior to application, essentially the same bond strengths are expected to be observed. Sirolimus elution may be tested by placing the sample that includes the bioadhesive layers into a glass vial filled with 25 ml phosphate buffered isotonic saline (PBS: pH 7.4; 37° C.). At predetermined intervals the buffer solution may be removed and the is vial refilled with fresh PBS. The sirolimus in the removed PBS is then extracted by mixing with 1:1 chloroform. The chloroform is separated and filtered through a polyethylene Frit and YLON+GL0.45 μm filter (Millipore). The released amount of sirolimus may be determined in triplicate by UV spectroscopy at 280 nm and compared to a standard calibration curve.

### EXAMPLE VII

### A Foam Cream

This example describes a composition for a pharmaceutical foam cream containing sirolimus.

The cream is produced by combining the following ingredients in a turbo diffuser: sirolimus 1%; white vaseline 12%; liquid paraffin 74%; white wax 3%; hydrogenated castor oil 5%; and methylglucose dioleate 5%. The operation of the turbo diffuser will first melt together the vaseline, paraffin, glucose and wax components by warming to a temperature of 72° C. while slowly stirring. Then hydrogenated castor oil is added to the mixture, which is then homogenized with a central turbo homogenizer. After cooling to room temperature, sirolimus is added to the mixture and then homogenized with the turbo diffuser under a light vacuum of 500 mm of mercury. The resulting cream is filled into suitable containers.

### EXAMPLE VIII

### A Foam Cream Canister

This example describes the filling requirements and composition for a sirolimus foam cream application canister.

Composition Within Each Canister

| | |
|---|---|
| Sirolimus | 10 mg |
| Cetyl stearyl alcohol USP | 160 mg |
| Mineral oil USP | 3640 mg |
| Mixture of n-butane/propane/isobutane | 150 mg |
| 55:25:20 (Purifair ™ 3.2) | |

In a first stainless steel container having an external jacket for warming, and a stirring blade, 3.2 kg cetyl stearyl alcohol (USP) is melted in 43.8 kg mineral oil (USP) to a temperature of 65°±0.5° C. while stirring. In a second stainless steel turbo vacuum diffuser provided with a water jacket for heating and cooling, a stirring blade, scraper and central turbo homogenizer, 29 kg mineral oil (USP) and 4 kg sirolimus foam cream made in accordance with Example VII are placed together. These two components are mixed by stirring at a low rate for 30 minutes under a light vacuum (500 mmHg). Thereafter, the above cetyl stearyl alcohol in mineral oil solution is cooled to 45° C. and added to the sirolimus/mineral oil mixture with continuous stirring under light vacuum for an additional 10 minutes while cooling the mixture to room temperature. The mixture is then subdivided by means of a filling machine into approximately 20,000 canisters. The canisters are thereafter closed with a polyethylene valve and for filling with propellant gas Purifair™ 3.2 and a polyethylene tube is inserted in the valve to facilitate complete delivery when the valve is depressed.

### EXAMPLE IX

### An Elastomeric Foam

This example describes the production of a sirolimus foam scaffolding composition.

A random copolymer of c-caprolactone-glycolide (PCL/PLGA) with a 35/65 molar composition is synthesized by a ring-opening polymerization reaction. Bezwada et al., Elastomeric Medical Device. U.S. Pat. No. 5,468,253 (herein incorporated by reference). A diethylene glycol initiator is added and is adjusted to a concentration of 1.15 mmole/mole of monomer to obtain a dried polymer having the following characteristics: i) an inherent copolymer viscosity of 1.59 dL/g in hexafluoroisopropanol at 25° C.; ii) a PCL/PGA molar ratio of 35.5/64.5 by proton NMR with about 0.5% residual monomer; iii) a glass transition and melting point of approximately −10° C. and 65° C., respectively.

A 5% (w/w) 35/65 PCL/PGA polymer/1,4-dioxane solution containing a desired concentration of sirolimus is next prepared by gentle heating to 60±0.5° C. and continuously stirring for at least 4 hours but not more than 8 hours. The solution is prepared in a flask with a magnetic stir bar. A clear homogeneous solution is then obtained by filtering the solution through an extra coarse porosity filter (i.e., a Pyrex brand extraction thimble with fritted disc) using dry nitrogen.

The solution is thereafter lyophilized, using for example, a laboratory scale lyophilizer-Freezemobile 6 (Virtis™). The freeze-dryer is preset at 20° C. under a dry nitrogen atmosphere and allowed to equilibrate approximately 30 minutes. The PCL/PGA polymer solution is poured into the molds just before the actual start of the cycle. A glass mold is preferred but a mold made of any material that is: i) inert to 1,4-dioxane; ii) has good heat transfer characteristics; and iii) has a surface that enables the easy removal of the foam. The best results are expected with a glass mold or dish weighing 620 grams, having optical glass 5.5 mm thick, and being cylindrical with a 21 cm outer diameter and a 19.5 cm inner diameter. Next the following steps are followed in a sequence to make 2 mm thick foam pieces:

i) The glass dish with the solution is carefully placed (without tilting) on the shelf of the lyophilizer, which is maintained at 20° C. The cycle is started and the shelf temperature is held at 20° C. for 30 minutes for thermal conditioning.

ii) The solution is then cooled to −5° C. by cooling the shelf to −5° C.

iii) After 60 minutes of freezing at −5° C., a vacuum is applied to initiate primary drying of the dioxane by sublimation; approximately one hour of primary drying under vacuum at −5° C. is needed to remove most of the solvent. At the end of this drying stage the vacuum level will typically reach about 50 mTorr or less.

iv) Next, secondary drying under a 50 mTorr vacuum or less is performed in two stages to remove the adsorbed dioxane. In the first stage, the shelf temperature is raised to +5° C. for approximately 1 hour. In the second stage, temperature is raised to 20° C. for approximately 1 hour.

v) At the end of the second stage, the lyophilizer is brought to room temperature and the vacuum is broken with nitrogen. The chamber is then purged with dry nitrogen for approximately 30 minutes before opening the door.

As one skilled in the art would know, the conditions described herein are typical and operating ranges depend on several factors e.g.: concentration of the solution; polymer molecular weights and compositions; volume of the solution; mold parameters; machine variables like cooling rate, heating rates; and the like. The above described process is expected to result in elastomeric foams having a random microstructure.

### EXAMPLE X

### Spray Application by a Catheter

This example provides a method and a device to administer sirolimus in an appropriate vehicle, as described herein, as a spray during an endoscopic procedure using an accompanying catheter.

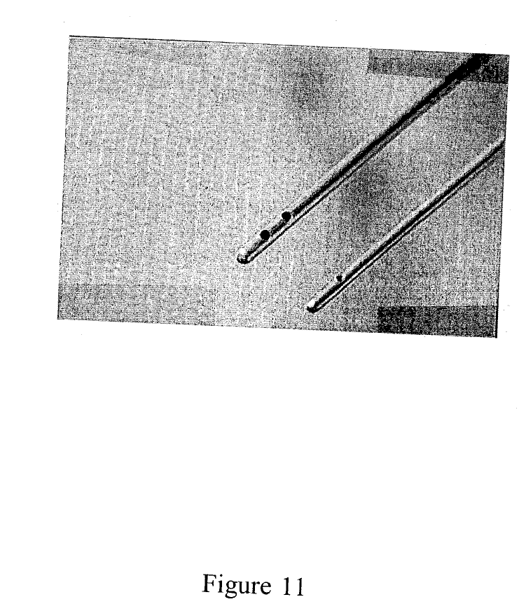

A “side hole catheter” has tiny round side holes cut into the catheter near a closed distal end. (e.g., FIG. 11) This catheter is constructed of a flexible, elongated, biocompatible polymer tubing which is hollow and thin-walled and should have a uniform diameter of 2 to 20 French, but preferably 5 to 10 French. Radioopaque markings on the catheter allows easy tracking of the catheter position via fluoroscopy. The catheter contains a medium comprising sirolimus. In practice, the catheter is inserted into a lumen of an endoscope system in accordance with standard approved procedures, and is moved carefully such that distal end of the catheter is positioned into or near the application site. A pharmaceutical solution of sirolimus is then injected under gentle pressure from a syringe-like reservoir attached to a female Luer lock and is impelled toward distal end of the catheter, emerging through the side holes and onto the application site. Alternately, a spray can or other apparatus under pressure may be attached to the Luer lock and spray administered via the side holes.

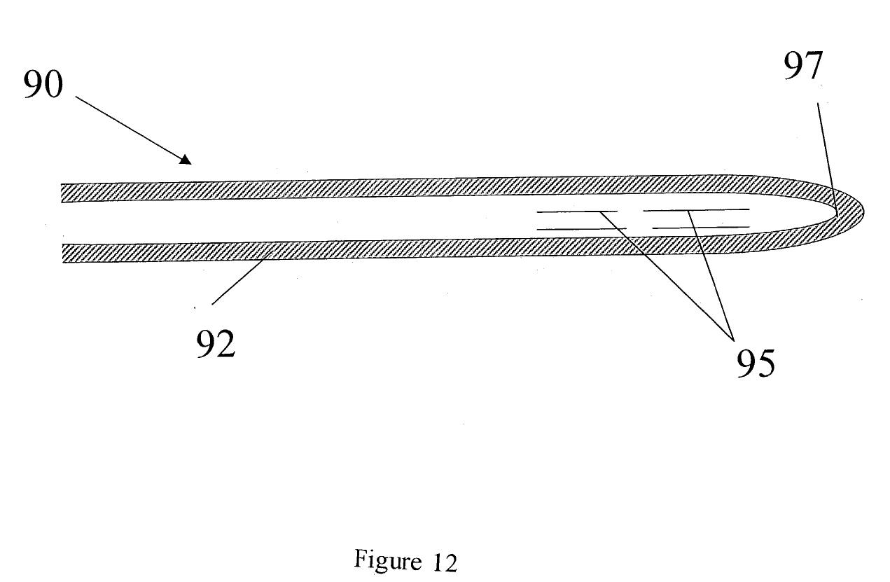

Alternatively, a “slit catheter” (FIG. 12), also composed of a flexible catheter 90 comprising a hollow, thin-walled, biocompatible polymer material 92 into which extremely thin slits 95 that are laser cut at regular intervals near a closed distal end 97. These slits are tight enough that infusate will not escape unless the fluid pressure within the catheter reaches a critical point that cause the slits to distend simultaneously and temporarily open. This catheter also contains exterior radiopaque markers to assist in the positioning of the device.

These slit catheters are also used in conjunction with an automated, piston-driven, pulsed infusion devices that are capable of delivering low volume regulated pulses of drug infusion at the proximal end of the catheter. When a pulse is delivered, the pressure within the catheter rises momentarily thus causing the slits to open momentarily to administer the sirolimus. Slit catheters are preferable to side hole catheters since, in the former type, the spray is delivered uniformly through all slits along the entire length of the catheter, whereas sprays from a “side hole” catheter are administered mainly from the most proximal side holes.

### EXAMPLE XI

### Spray Application by a Single Dose Dispenser

This example describes a single dose spray dispenser that is capable of applying a single dose of sirolimus, tacrolimus and analogs of sirolimus. It is understood by one skilled in the art that the basic concept described below may be modified and adapted to administer a single dose either internally or externally. For example, the dispenser described below may be reconfigured for operation with a catheter for administration to an intraluminal site within the body. Depending upon the size of the surgical site, a medical practitioner may dispense one or more cans at any one particular site.

The Dispenser Device

A dispenser device for spraying a single dose of sirolimus intended to cover about 50 cm² of wound at the rate of about 200 μg/cm² will have a cylinder containing a predetermined dose of a liquid medium containing sirolimus, tacrolimus or an analog of sirolimus. A piston will slide in a sealed manner within the cylinder between a storage position in which it isolates the cylinder to an actuated position. An outlet passage will connect the cylinder to an outlet orifice where the entire single dose of liquid is expelled from the device when the piston is slid from the storage to the actuated position. Martin et al., Device For Dispensing A Single Dose Of Fluid. U.S. Pat. No. 6,345,737 (herein incorporated by reference).

The Liquid Medium

Sirolimus will be dissolved in olive oil at a concentration of 1 mg/ml. Alternatively, soluble monoacyl and diacyl derivatives of sirolimus are prepared according to known methods. Rakhit, U.S. Pat. No. 4,316,885 (herein incorporated by reference). These derivatives are used in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like. Furthermore, water soluble prodrugs of sirolimus may be used including, but not limited to, glycinates, propionates and pyrrolidinobutyrates. Stella et al., U.S. Pat. No. 4,650,803 (herein incorporated by reference).

Controlled Release

Alternatively, the liquid media described above is prepared using microspheres prepared according to Example I or using liposomes prepared according to Example III.

### EXAMPLE XII

### Aerosolizaton

This example describes one method of providing a sirolimus aerosol spray to an area of interest.

The Nebulizer

A nebulizer will transform solutions or suspensions of sirolimus according to any of the applicable Examples discusses herein, into a therapeutic aerosol mist either by means of acceleration of a compressed gas, typically air or oxygen, through a narrow venturi orifice. In particular, embodiments of sirolimus media exhibiting controlled release capabilities are preferred. Sirolimus is present in a liquid carrier in an amount of up to 5% w/w, but preferably less than 1% w/w of the formulation. The carrier is typically water or a dilute aqueous alcoholic solution, preferably made isotonic with body fluids by the addition of, for example, sodium chloride. Solubility enhancing agents are well known in the art and may be added as deemed required depending upon the required concentration. Optional additives include preservatives if the formulation is not prepared sterile, for example, methyl hydroxybenzoate, antioxidants, volatile oils, buffering agents and surfactants.

The present invention contemplates the use of many devices to generate an aerosol and the following exemplary device is not intended to limit the invention. The nebulizer device has a lever that activates an air spring-valve joint directly connected to an external source of pressurized air or other gaseous propellant such that the air enters an air chamber. An air channel will extend from an air chamber to the distal end of the aerosolization apparatus. The air channel terminates into a rod extension that contains the aperture aerosolization tips.

A fluid chamber tip also include apertures that communicate with the air channels. When the sirolimus fluid chamber tip end is inserted into the air channel aperture, no air passes out of air chamber. When the fluid chamber tip end is, however, withdrawn from the air channel aperture, air and fluid mix into an aerosol and exit the apparatus through a dispensing tip.

The Liquid Medium

Sirolimus will be dissolved in olive oil at a concentration of at least 10 mg/ml. Alternatively, soluble monoacyl and diacyl derivatives of sirolimus are prepared according to known methods. Rakhit, U.S. Pat. No. 4,316,885 (herein incorporated by reference). These derivatives are used in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like. Furthermore, water soluble prodrugs of sirolimus may be used including, but not limited to, glycinates, propionates and pyrrolidinobutyrates. Stella et al., U.S. Pat. No. 4,650,803 (herein incorporated by reference).

### EXAMPLE XIII

### A Multiple Lumen Catheter

This example describes a catheter capable of coadministration of several sirolimus solutions simultaneously, or mixing a sirolimus solution with a non-sirolimus solution into a single composition. Specifically contemplated is the mixing of two separate components in order to spray a sirolimus-containing bioadhesive.

A device for applying two-component products, such as medical tissue bioadhesive, has a flat head piece connected at the front end to a tubular body. A multiple lumen tube is therewith expected to be in communication with a tubular body. The dorsal surface of the head piece also has portions of two cannula hubs. A multiple lumen tube is comprised of three lumina which extend in parallel from the inner end of the lumen tube to the discharge end. Two lumina are connected to each of two syringes (respectively), either barrels of which may contain a sirolimus-containing composition. The plunger rods of the syringes are coupled by a bridging member such that both are operated simultaneously to permit equal mixing and administration of the compositions in both barrels.

Two of the cannula hubs, partially included in the head piece, are connected to rigid cannulas preferably made of metal. The two metal cannulas are oriented in the head piece such that they extend in V-shape. A third lumen is an end of a connecting tubule and is connected to a soft flexible air tube. An air tube also extends from the tip of the V formed by the two metal cannulas straight to the rear end of the head piece.

The air tube is in direct communication with the third lumen of the multiple lumen tube through the connecting tubule. The precise flow of the compositions from the two syringe barrels and the air flow is expected to emerge from the catheter close together as a thin jet from the discharge end of the multiple lumen tube. The compositions from the two syringe barrels are sprayed in an optimal mixture by the air flow so that the treated site is supplied with a sufficient quantity of dispersed sirolimus bioadhesive. Due to the separate transport of the compositions from the two syringe barrels and the air in different lumens, the compound containing material is only mixed when past the discharge end of the multiple lumen tube. Accordingly, the portions of the compound containing material from the two syringe barrels are dosed exactly and the composition of a sirolimus bioadhesive is always correct.

### EXAMPLE XIV

### Bioadhesive Applicator Device

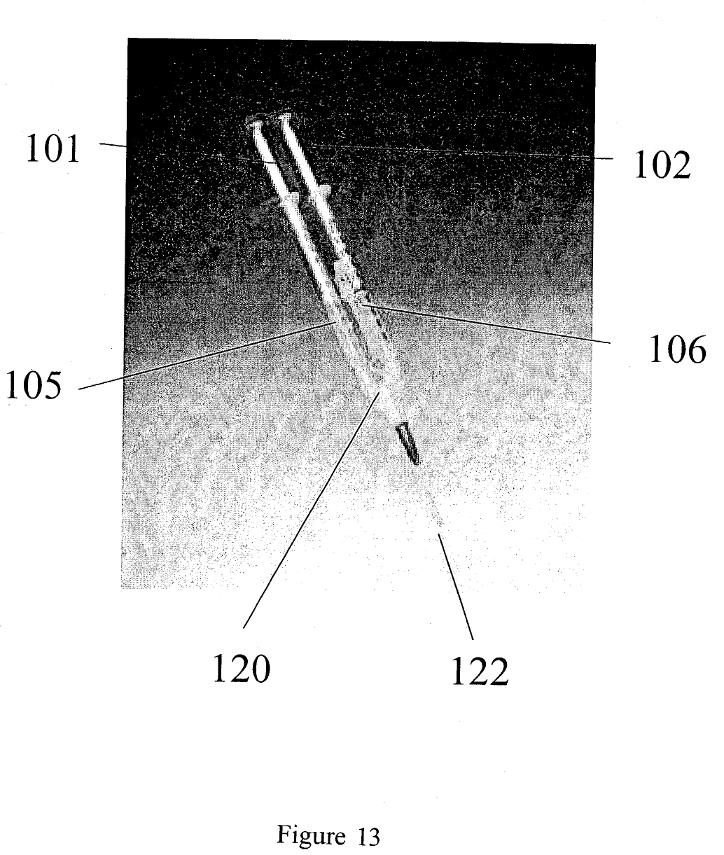

This example describes one embodiment of a bioadhesive applicator device (see FIG. 13).

The applicator is constructed as a pair of syringes 105 and 106, each of which has plungers 101 and 102 which variably slide within a hollow of each respective syringe body between a fully retracted position to a fully compressed position. Each of the syringes 105 and 106, respectively, contain a different material (i.e., for example, thrombin versus fibrin) that, become an adhesive compound when mixed. The syringes merge into a common mixing area 120 at one end, wherein the mixing area 120 is adapted to connect with each outlet of syringes 105 and 106. The plungers 101 and 102 will push the respective medium out of each syringe 105 and 106, whereupon mixing occurs prior to exiting from a nozzle 122 as a single stream. After the mixed adhesive medium exits the applicator, the mixture will harden into a bioadhesive onto the target tissue site.

We claim:

1. A drug attached to a carrier, the drug being selected from the group consisting of sirolimus, tacrolimus, everolimus and the analogs and derivatives thereof, the carrier onto which the drug is attached being selected from the group consisting of microparticles, gels, xerogels, bioadhesives, foams and liquids.

2. The drug attached to a carrier of claim 1, wherein the carrier comprises a biocompatible material.

3. The drug attached to a carrier of claim 1, wherein the carrier comprises a biodegradable material.

4. The drug attached to a carrier of claim 1, wherein the microparticles are selected from the group consisting of microspheres, microencapsulating particles, microcapsules and liposomes.

5. The microparticle of claim 4 comprising a polymer selected from the group consisting of poly(lactide-co-glycolide), aliphatic polyesters, poly-glycolic acid, poly-lactic acid, hyaluronic acid, modified polysacchrides, poly(ethylene oxide), lecithin and phospholipids.

6. The drug attached to a carrier of claim 1, wherein the carrier comprises a material selected from the group consisting of poly(lactide-co-glycolide), aliphatic polyesters, poly-glycolic acid, poly-lactic acid, hyaluronic acid, modified polysacchrides, poly(ethylene oxide), lecithin, phospholipids, fibrin sealants, polyethylene oxide, polypropylene oxide, block polymers of polyethylene oxide and polypropylene oxide, polyethylene glycol, methacrylates and cyanoacrylates.

7. The drug attached to a carrier of claim 1, wherein the carrier releases said drug in a controlled release manner.

8. The drug attached to a carrier of claim 1, wherein the carrier is colored.

9. A medium, comprising a compound selected from the group consisting of sirolimus, tacrolimus, analogs of sirolimus and pharmaceutically acceptable salts thereof, wherein said medium is selected from the group consisting of microparticles, gels, xerogels, bioadhesives and foams.

10. The medium of claim 9, wherein said medium comprises a biocompatible material.

11. The medium of claim 9, wherein said medium comprises a biodegradable material.

12. The medium of claim 9, wherein said microparticles are selected from the group consisting of microspheres, microencapsulating particles, microcapsules and liposomes.

13. The medium of claim 9, wherein said medium is colored.

14. The medium of claim 9, wherein said analog of sirolimus is selected from the group consisting of everolimus, CCI-779, ABT-578, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin and 2-desmethyl-rapamycin.

15. The medium of claim 9, further comprising a second compound selected from the group consisting of antiinflammatory, corticosteriods, antithrombotics, antibiotics, antivirals, analgesics and anesthetics.

16. A device, said device comprising a reservoir comprising the medium of claim 9 and capable of delivering said medium of claim 9 to a surgical site.

17. The device of claim 16, wherein said delivering is in the form of a spray.

18. The device of claim 16, wherein said delivering is in the form of an aerosol.

19. The device of claim 16, wherein said device comprises a catheter.

20. The device of claim 16, wherein said device is configured for endoscopic surgery.

21. The device of claim 16, wherein said device is configured for fluoroscopic surgery.

22. A medical device wherein at least a portion of said device is coated with the medium of claim 9.

23. A method, comprising:
a) providing:
i) a medium comprising a compound selected from the group consisting of sirolimus, tacrolimus, analogs of sirolimus and pharmaceutically acceptable salts thereof, wherein said medium is selected from the group consisting of microparticles, gels, xerogels, bioadhesives and foams; and
ii) a surgical site of a patient;
b) contacting said surgical site with said medium.

24. The method of claim 23, wherein said surgical site comprises a closed surgical site.

25. The method of claim 23, wherein said medium of step (a) is housed in a device.

26. The method of claim 25, wherein said medium of step (b) contacts said surgical site in the form of a spray.

27. The method of claim 26, wherein said spray in the form of an aerosol.

28. The method of claim 25, wherein said device comprises a catheter.

29. The method of claim 25, wherein said device is configured for endoscopic surgery.

30. The method of claim 29, wherein said catheter delivers medium to a closed surgical site.

31. The method of claim 25, wherein said device is configured for fluoroscopic surgery.

32. The method of claim 23, wherein said medium comprises a biocompatible material.

33. The method of claim 23, wherein said medium comprises a biodegradable material.

34. The method of claim 23, wherein said microparticles are selected from the group consisting of microspheres, microencapsulating particles, microcapsules and liposomes. said microparticle is a microsphere.

35. The method of claim 23, wherein said medium is colored.

36. The method of claim 23, wherein said medium further comprises a second compound selected from the group consisting of antiinflammatory, corticosteriods, antithrombotics, antibiotics, antivirals, analgesics and anesthetics.

37. A collection of microspheres comprising a biocompatible material for placement at or near the site of a surgical procedure to reduce the formation of scar tissue and adhesions, the microspheres having a diameter between 0.1 and 100 microns and a cytostatic and antiproliferative drug attached to the microspheres that is adapted for release over time.

38. A method for delivering a gel or liquid comprising microparticles having an attached compound selected from the group consisting of sirolimus, tacrolimus and analogs of sirolimus to a surgical site.

39. A gel or liquid, comprising at least one compound selected from the group consisting of sirolimus, tacrolimus, analogs of sirolimus and pharmaceutically acceptable salts thereof, wherein said compound is attached to a microparticle.
